SEQUENCE LISTING (7 sequences)

SEQ ID NO: 1 - protein, 90 aa, synthetic construct
DDDGKTVDGPSPLVLRAFINGVNNGRNGLGSIYVFASGNGGIYEDNSNFDGYANSVFTITIGGIDKHGKRPKYSEASSSQLAVTYAGGSA

SEQ ID NO: 2 - protein, 90 aa, synthetic construct
PDDGKTMEGPDILVLRAFINGVQNGRDGKGSIYVFASGNGGGFEDNSNFDGYTNSIYSITVGAIDRKGLHPSYSEASSAQLVVTYSSGSG

SEQ ID NO: 3 - protein, 90 aa, unidentified
DDDGKTVDGPSPLVLRAFINGVNNGRNGLGSIYVFASGNGGIYDDNCNFDGYANSVFTITIGGIDKHGKRFAYSEACSSQLAVTYAGGSA

SEQ ID NO: 4 - DNA, 270 bp, unidentified
  source 1..270: mol_type = unassigned DNA
gatgacgatggaaaaaccgttgatgggccttctcctcttgttcttagagcatttattaatggagtaaataatgggaggaatgggttgggttctatctatgtttttgcatcaggaaatggcggaatatacgatgacaactgtaattttgatggatatgcaaatagcgtgtttactattactattggtggtatagataaacacggaaagcgctttgcatattctgaagcgtgttcttctcagttagctgttacatatgcaggcggaagtgca

SEQ ID NO: 5 - protein, 844 aa, unidentified
MRFLGSIALVLSSISVASANVRSRSYDTHEFFALHLDDSASPSHVAQLLGARHEGQIGELANHHTFSIPRERSSDLDALLERARAARKIRRRARDDATSQEQHNDALGGILWSQKLAPKKRLVKRVPPPERLARTFATGKEDPVAAQSQKRIASTLGITDPIFNGQWHLFNTVQLGHDLNVTGVWMEGITGKGVTTAVVDDGLDMYSNDLKPNYFPEGSYDFNDHTPEPRPRLSDDKHGTRCAGEIAAARNDVCGVGVAYDSRVAGVRILSKAIDDADEATAINFAYQENDIFSCSWGPPDDGATMEGPGILIKRAFVNGVQNGRGGKGSIFVFAAGNGASFEDNCNFDGYTNSIYSITVGAIDREGNHPSYSESCSAQLVVAYSSGSGDAIHTTDVGTDKCYSFHGGTSAAGPLAAGTVALALSARPELTWRDAQYLMVETAVPIHEDDGSWQVTKAGRKFSHDWGYGKVDAYALVQKAKTWELVKPQAWFHSPWLRVQHKVPQGDQGLASSYEVTEQMMKNANIARLEHVTVTMNVNHTRRGDLSVELRSPEGIVSHLSTTRKSDNEKAGYVDWTFMTVAHWGESGVGRWTVIVKDTNVNEFTGEFIDWRLNLWGEAIDGANQKPHPFPDEHDDDHSIEDAIVATTSVETGPTKTGVPGSTDDTINRPVNAKPVETQTPSPAETTATKLAPPAETRPAATATSSPTPPAASDSFLPSFMPTFGASKRTQIWIYAAIGSIIVFCIGLGIYFQVQRRKRILNNPRDDYDFEMIEDENALHGGNGRSGRTQRRGGELYNAFAGESDEEEPLFSDEDDEPYRDRAPSEDRLRDTSSDDRSLRHGDH

SEQ ID NO: 6 - protein, 924 aa, Candida albicans
MLPIKLLIFILGYLLSPTLQQYQQIPPRDYENKNYFLVELNTTNSQKPLIDFISHYRGHYNFEHQLSSLDNHYVFSIDKSHPHNSFLGNHNSNEYNLMKRQLGHEQDYDELISHVESIHLLPMKKLSKRIPVPIEMEDVVFDNRDDTGSDNHEATDEAHQKLIEIAKKLDIHDPEFTTQWHLINLKYPGHDVNVTGLWLEDILGQGIVTALVDDGVDAESDDIKQNFNSEGSWDFNNKGKSPLPRLFDDYHGTRCAGEIAAVKNDVCGIGVAWKSQVSGIRILSGPITSSDEAEAMVYGLDTNDIYSCSWGPTDNGKVLSEPDVIVKKAMIKGIQEGRDKKGAIYVFASGNGGRFGDSCNFDGYTNSIYSITVGAIDYKGLHPQYSEACSAVMVVTYSSGSGEHIHTTDIKKKCSATHGGTSAAAPLASGIYSLILSANPNLTWRDVQYISVLSATPINEEDGNYQTTALNRKYSHKYGYGKTDAYKMVHFAKTWVNVKPQAWYYSDIIEVNQTITTTPEQKAPSKRDSPQKIIHSSVNVSEKDLKIMNVERVEHITVKVNIDSTYRGRVGMRIISPTGVISDLATFRVNDASTRGFQNWTFMSVAHWGETGIGEWKVEVFVDDSKGDQVEINFKDWQFRIFGESIDGDKAEVYDITKDYAAIRRELLEKEKQNSKSTTTTSSTTTATTTSGGEGDQKTTTSAENKESTTKVDNSASITTSQTASLTSSNEQHQPTESNSDSDSDTDDENKQEGEEDNDNDNDNGNKKANSDNTGFYLMSIAVVGFIAVLLVMKFHKTPGSGRRRRRRDGYEFDIIPGEDYSDSDDDEDDSDTRRADDDSFDLGHRNDQRVVSASQQQRQYDRQQDEARDRLFDDFNAESLPDYENDMFKIGDEEEEEEEEEEGQQSAKAPSNSEGNSGTSTKK

SEQ ID NO: 7 - protein, 917 aa, Cryptococcus neoformans
MRTLLSLWGILLALIVPPSLALQRPQPRSYDTHAYYALELDPSISPAAALQLSKSLGVELVERIGELDGHWLVRTEGWTPEHASITKRSVSHDPILKRWEALPSSLGKKSLTPLSLKQRAKRHKSYSPRSRHSRDDRTELLYAQNELHLADPMLDQQWHLINTQMKDIELNVTGLWGRGITGEGVHVVIIDDGLDVESKDLKDNFFAEGSYDFNDHTELPIPRLKDDQHGTRCAGEIAAVPNDVCGVGVAYDSKIAGVRILSAPISDADEAAALNYAYQLNDIYSCSWGPPDDGRSMEAPDGLILKAMVNGVQKGRDGKGSVFVFAAGNGGGSDDQCNFDGYTNSIFSVTVGAVDRKGLHPYYSEMCAAMMVVAPSSGSGDHIHTTDVGKDKCSHSHGGTSAAAPLAVGVFALALSVRPDLTWRDIQHLAVRHAVFFNPDDPAWELTAAGRHFSYKYGYGKLDAGLFVEAAEKWQLVKPQTWYDSPSVYLPTTSPADVTRRQDEAADGPTSSDEETSNPPPVVEPSGSFITEDGVISTYEVTQSMLFDANFERLEHVTVRVWIDHQRRGDVEVELTSPNGVVSVLCRQRRFDNADSGFPGWKFMSLKHWDENPVGTWTIKVKDQVNPDKTGRFVAWSLQLWGESVDPALAKLWAPAEEGQPDEEQTGSNPSTTVSQKPKPTALLPGDHGEASGEATQPGLGSATAHPQPTSTTGDAGNVAEPTGPTDADADEGFFSGISNLASSSTWLAGAGAIIILSGAAIGAFFFIRARRQKRNLFGLSNNGQGARGAYEPVDDVQMSLLERGRRKFGKSKSESQGTKDLYDAFGDGPSDEEEEDLDERTALRYHDGFLEDDEPNEVGPKTEYKDEPESEPETFKDGEETVGTKDKGKGKGPSEGESGSGSSSSWQDAADEEARV

(19) United States
(12) Patent Application Publication
(10) Pub. No.: US 2025/0213663 A1
(43) Pub. Date: Jul. 3, 2025

(54) IMMUNOGENIC ANTI-FUNGAL POLYPEPTIDES, COMPOSITIONS AND METHODS THEREOF

(71) Applicant: University of Georgia Research Foundation, Inc., Athens, GA (US)

(72) Inventors: Karen A. Norris, Athens, GA (US); Whitney Rabacal, Athens, GA (US); Emily Rayens, Athens, GA (US)

(73) Assignee: University of Georgia Research Foundation, Inc., Athens, GA (US)

(21) Appl. No.: 19/085,932

(22) Filed: Mar. 20, 2025

Related U.S. Application Data
(63) Continuation of application No. PCT/US2023/074756, filed on Sep. 21, 2023.
(60) Provisional application No. 63/409,491, filed on Sep. 23, 2022.

(51) Int. Cl. A61K 39/00; A61K 38/00; A61P 31/10; C07K 16/40; C12N 9/64

(52) U.S. Cl. CPC A61K 39/0002; A61P 31/10; C07K 16/40; C12N 9/6408; A61K 38/00; A61K 2039/575; C12Y 304/21061

(57) ABSTRACT

Described and featured are immunogenic compositions comprising non-naturally occurring pan-fungal kexin peptides and methods of using such compositions for the treatment or prevention of various diseases or severe diseases, and/or the symptoms thereof, particularly those associated with infection by fungal pathogens (e.g., Pneumocystis, Aspergillus, Candida, or Cryptococcus) in the subject. The immunogenic peptides or immunogenic fragments thereof may be recombinantly produced, recombinant, and/or isolated.

### CROSS REFERENCE TO RELATED APPLICATION

This application is a continuation under 35 U.S.C. § 111 (a) of PCT International Patent Application No. PCT/US2023/074756, filed Sep. 21, 2023, designating the United States and published in English, which claims priority to and the benefit of U.S. Provisional Application No. 63/409,491, filed Sep. 23, 2022, the entire contents of each of which are incorporated by reference herein.

### STATEMENT OF GOVERNMENT SUPPORT

This invention was made with government support under grant number R01 AI148365 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Sep. 28, 2023, is named “173093-011602US_SL” and is 10,307 bytes in size.

### BACKGROUND

Pathogenic fungal organisms are universal in the environment and can cause global health threats. Pathogenic fungi are typically not harmful to individuals with healthy and normally functional immune systems even after exposure, for example, by inhalation. However, individuals with weakened or compromised immune systems, or those having pre-existing medical conditions, such as those with lung diseases or viral infections, such as HIV/AIDS infection, are at a higher risk of developing serious health problems and adverse reactions following exposure to and infection by fungal pathogens.

Because of the grave repercussions of infection by fungal organisms in individuals in poor medical health and in those with weakened immune systems, there is an ongoing and urgent need for methods and compositions for treating or preventing infection and associated diseases caused by these pathogens. Cost-effective and efficient methods of treatment of and protection from fungal pathogens are required, particularly in less affluent parts of the world. Immunogens with improved characteristics for use in methods of treating or preventing fungal pathogen-associated diseases for which suitable therapies are currently nonexistent or inadequate are particularly desirable in view of the ever-present health threats that fungal pathogens pose to at-risk individuals worldwide.

### SUMMARY

Immunogens, compositions and methods for treating or preventing disease associated with or caused by infection (e.g., opportunistic infection) by fungal pathogens, and for treating or preventing pulmonary disease and poor pulmonary function associated with infection by fungal pathogens, as well as other diseases and/or the symptoms thereof, are provided and described herein.

In an aspect, an immunogenic peptide, or a polynucleotide encoding the immunogenic peptide comprising the amino acid sequence (in an amino terminus (NH₂) to carboxy terminus (COOH) orientation):
1 DDDGKTVDGPSPLVLRAFINGVNNGRNGLGSIYVFASGNGGIYEDNSNFD
51 GYANSVFTITIGGIDKHGKRPKYSEASSSQLAVTYAGGSA, (SEQ ID NO: 1), (called “Pan-fungal peptide 1b” or “PF-KEX1b” peptide herein), is provided. In an embodiment, a functional fragment of the PF-KEX1b immunogenic peptide is provided.

In an aspect, an immunogenic peptide, or a polynucleotide encoding the immunogenic peptide comprising the amino acid sequence (in an amino terminus (NH₂) to carboxy terminus (COOH) orientation):
1 PDDGKTMEGPDILVLRAFINGVQNGRDGKGSIYVFASGNGGGFEDNSNFD
51 GYTNSIYSITVGAIDRKGLHPSYSEASSAQLVVTYSSGSG, (SEQ ID NO: 2), (called “Pan-fungal peptide 2b” or “PF-KEX2b” peptide herein), is provided. In an embodiment, a functional fragment of the PF-KEX2b immunogenic peptide is provided.

In an aspect, an immunogenic peptide or a polynucleotide encoding an immunogenic peptide consisting of the amino acid sequence:
1 DDDGKTVDGPSPLVLRAFINGVNNGRNGLGSIYVFASGNGGIYEDNSNFD
51 GYANSVFTITIGGIDKHGKRPKYSEASSSQLAVTYAGGSA, (SEQ ID NO: 1), Pan-fungal peptide 1b or PF-KEX1b, is provided.

In an aspect, an immunogenic peptide or a polynucleotide encoding an immunogenic peptide consisting of the amino acid sequence:
1 PDDGKTMEGPDILVLRAFINGVQNGRDGKGSIYVFASGNGGGFEDNSNFD
51 GYTNSIYSITVGAIDRKGLHPSYSEASSAQLVVTYSSGSG, (SEQ ID NO: 2), Pan-fungal peptide 2b or PF-KEX2b, is provided.

In an aspect, an immunogenic peptide or a polynucleotide encoding an immunogenic peptide having at least 98% sequence identity to the amino acid sequence:
DDDGKTVDGPSPLVLRAFINGVNNGRNGLGSIYVFASGNGGIYEDNSNFD
GYANSVFTITIGGIDKHGKRPKYSEASSSQLAVTYAGGSA (SEQ ID NO: 1), (PF-KEX1b), wherein the peptide contains a serine(S) amino acid residue at least at positions 47 and 77 of the amino acid sequence, is provided.

In an aspect, an immunogenic peptide or a polynucleotide encoding an immunogenic peptide having at least 98% sequence identity to the amino acid sequence:
PDDGKTMEGPDILVLRAFINGVQNGRDGKGSIYVFASGNGGGFEDNSNFD
GYTNSIYSITVGAIDRKGLHPSYSEASSAQLVVTYSSGSG (SEQ ID NO: 2), (PF-KEX2b), wherein the peptide contains a serine(S) amino acid residue at least at positions 47 and 77 of the amino acid sequence, is provided.

In an embodiment of any of the above-delineated aspects, an immunogenic peptide of the immunogenic polypeptide or an immunogenic fragment of the immunogenic peptide is provided. In an embodiment of any of the above-delineated aspects and embodiments thereof, the immunogenic PF-KEX1b and PF-KEX2b peptides, or an immunogenic portion thereof, may afford benefits in connection with manufacturing and formulation processes. By way of nonlimiting example, the peptides are stable and not expected to be subject to cross-linking and/or aggregation. In an embodiment of any of the above-delineated aspects and/or embodiments thereof, the immunogenic polypeptide, the immunogenic peptide of the immunogenic polypeptide, or an immunogenic fragment of the immunogenic polypeptide or peptide is recombinant and/or recombinantly produced. In an embodiment of any of the above-delineated aspects and/or embodiments thereof, the immunogenic polypeptide, the immunogenic peptide of the immunogenic polypeptide, or an immunogenic fragment of the immunogenic polypeptide or peptide is isolated. The terms “polypeptide” and “peptide” are also used interchangeably herein.

In some embodiments, a polypeptide or a functional peptide thereof, or a peptide or a functional fragment thereof, has at least 85%, at least 95%, at least 96%, at least 97%, at least 97.5%, at least 98%, at least 98.5%, at least 99%, at least 99.5%, or 100% amino acid sequence identity to the sequence set forth in SEQ ID NO: 1 or SEQ ID NO: 2.

In another aspect, an immunogenic composition is provided which comprises an effective amount of an immunogenic peptide or a polynucleotide encoding an immunogenic peptide described supra and a pharmaceutically acceptable carrier, vehicle, or excipient. In some embodiments, the immunogenic composition further comprises an adjuvant. In an embodiment, the adjuvant is alpha-galactosylceramide (αGC), alum, or ALHYDROGEL®.

In another aspect, a method of eliciting an immune response in a subject is provided, in which the method involves administering to the subject the immunogenic peptide, encoding polynucleotide, or the immunogenic composition of any one of the above-delineated aspects and/or embodiments thereof.

In another aspect, a method of treating or protecting a subject against disease or a symptom thereof associated with or caused by a fungal infection is provided, in which the method involves administering to the subject the immunogenic peptide or encoding polynucleotide, or the immunogenic composition of any one of the above-delineated aspects and/or embodiments thereof, in an amount effective to treat or protect the subject against the fungal disease or a symptom thereof. In an embodiment, the peptide is recombinant, recombinantly produced, and/or isolated.

In another aspect, a method of treating or protecting a subject against disease, or a symptom thereof, associated with or caused by a fungal infection is provided, in which the method comprising administering to the subject an isolated antiserum comprising an antibody, or an antigen-binding fragment thereof, or an isolated or purified antibody, or an antigen-binding fragment thereof, that specifically binds to the immunogenic peptide of any one of the above-delineated aspects and/or embodiments thereof, in an amount effective to treat or protect the subject against the fungal disease or a symptom thereof. In an embodiment, the peptide is recombinant, recombinantly produced, and/or isolated. In some embodiments, the antibody is a monoclonal antibody, a polyclonal antibody, or an antigen-binding fragment thereof.

In some embodiments of any of the above-delineated methods and/or embodiments thereof, the disease or symptoms thereof, is associated with or caused by an Aspergillus, Candida, Pneumocystis, and/or Cryptococcus fungal pathogen. In an embodiment, the disease or symptom thereof is associated with or caused by an Aspergillus fumigatus fungal pathogen. In some embodiments, the methods treat pulmonary disease, pulmonary dysfunction, or a symptom thereof. In some embodiments, the pulmonary disease or pulmonary dysfunction is Pneumocystis pneumonia (PCP), aspergillosis, or Invasive Pulmonary Aspergillosis (IPA).

In another aspect, a method of treating or protecting a subject against disease or severe disease, or symptoms thereof, associated with or caused by fungal infection is provided, in which the method involves administering to the subject an immunogenic composition comprising the immunogenic peptide of SEQ ID NO: 1 or the immunogenic peptide of SEQ ID NO: 2, or an immunogenic fragment thereof, or a polynucleotide encoding the peptide of SEQ ID NO: 1 or of SEQ ID NO: 2, or a functional fragment thereof, in an amount effective to induce an immune response against one or more of Pneumocystis, Aspergillus, Candida, or Cryptococcus fungal pathogens. In an embodiment, the fungal pathogen is Aspergillus or Aspergillus fumigatus. In an embodiment, the peptide of SEQ ID NO: 1 or 2 is recombinant, recombinantly produced, and/or isolated.

In another aspect, a method of treating or protecting a subject against fungal infection is provided, in which the method comprises administering to the subject an effective amount of an antibody or an antigen-binding fragment thereof that specifically binds to the PF-KEX2b peptide of SEQ ID NO: 2 or to the PF-KEX1b peptide of SEQ ID NO: 1. In an embodiment, the PF-KEX2b peptide of SEQ ID NO: 2 or the PF-KEX1b peptide of SEQ ID NO: 1 is recombinant, recombinantly produced, and/or isolated. In an embodiment, the antibody or an antigen-binding fragment thereof is present in or is isolated from an antiserum derived from a donor subject. In some embodiments, the antibody or an antigen-binding fragment thereof specifically binds a Kex peptide of one or more Pneumocystis, Aspergillus, Candida, or Cryptococcus fungal pathogens.

In an embodiment of the method of any one of the above-delineated aspects and/or embodiments thereof, the subject is a mammal or a human (human patient). In some embodiments, the subject is immunocompromised or immunosuppressed. In some embodiments, the subject is a pre-transplant subject or a post-transplant subject. In some embodiments, the immunocompromised or immunosuppressed subject is being treated for an immunodeficiency disease (e.g., HIV, AIDS, HIV/AIDS), an autoimmune disease, rheumatoid arthritis, or psoriasis. In some embodiments, the disease, or symptom thereof, is associated with or caused by infection by an Aspergillus, Pneumocystis, Candida, and/or Cryptococcus fungal species. In an embodiment, the disease, or symptom thereof, is associated with or caused by infection by an Aspergillus fungal species. In other embodiments of the above-delinenated methods and/or embodiments thereof, the subject has, is at risk of having, or is susceptible to, a fungal infection by an Aspergillus, Pneumocystis, Candida, and/or Cryptococcus fungal species and disease or symptoms thereof associated with or caused by the infection. In an embodiment, the method treats or protects against pulmonary disease, pulmonary dysfunction, aspergillosis, Invasive Pulmonary Aspergillosis (IPA), or a symptom thereof.

In another aspect, a method of treating or protecting a subject against disease or severe disease, or a symptom thereof, is provided, in which the method involves administering to the subject the immunogenic peptide or polynucleotide or a pharmaceutical composition thereof, of any of the above-delineated aspects and/or embodiments thereof, in an amount effective to treat or protect the subject against the disease or severe disease, or a symptom thereof. In an embodiment, the peptide is recombinant, recombinantly produced, and/or isolated.

In another aspect, a method of treating or protecting a subject against disease or severe disease, or a symptom thereof, is provided, in which the method involves administering to the subject an isolated antiserum comprising an antibody, or an antigen-binding fragment thereof, that specifically binds an immunogenic peptide of any of the above-delineated aspects and/or embodiments thereof in an amount effective to treat or protect the subject against the disease or severe disease, or a symptom thereof.

In another aspect, a method of treating or protecting a subject against disease or severe disease, or a symptom thereof, is provided, in which the method involves administering to the subject an isolated or purified antibody or an antigen-binding fragment that specifically binds an immunogenic peptide of any of the above-delineated aspects and/or embodiments thereof, in an amount effective to treat or protect the subject against the disease or severe disease, or a symptom thereof. In an embodiment, the antibody is a monoclonal antibody, a polyclonal antibody, or an antigen-binding fragment thereof.

In embodiments of the above-delineated methods, the disease or severe disease, or a symptom thereof, is selected from pulmonary disease, asthma, severe asthma, refractory asthma, Chronic Obstructive Pulmonary Disease (COPD), chronic bronchitis, pneumonia, Pneumocystis pneumonia, bronchiectasis, aspergillosis, Invasive Pulmonary Aspergillosis (IPA), vaginitis, urinary tract infections (UTIs), organ transplant, tissue transplant, immunodeficiency disease, HIV, AIDS, HIV/AIDS, congenital disease, autoimmune disease, rheumatoid arthritis, psoriasis, inflammation-related disease, diabetes, Type 1 diabetes, or Type 2 diabetes.

In another aspect, a method of treating or protecting a subject against disease or severe disease, or symptoms thereof, associated with or caused by an Aspergillus fungal pathogen is provided, in which the method involves administering to the subject a PF-KEX2b peptide of SEQ ID NO: 2, or a polynucleotide encoding PF-KEX2b, or an immunogenic composition comprising PF-KEX2b peptide or a polynucleotide encoding PF-KEX2b, in an amount effective to induce an immune response and treat or protect against disease, severe disease, or symptoms thereof, associated with infection by the Aspergillus fungal pathogen. In an embodiment, the PF-KEX2b peptide of SEQ ID NO: 2 is recombinant, recombinantly produced, and/or isolated.

In another aspect, a method of treating or protecting a subject against disease or severe disease, or symptoms thereof, associated with or caused by an Aspergillus fungal pathogen, is provided, in which the method involves administering to the subject PF-KEX1b peptide of SEQ ID NO: 1, or a polynucleotide encoding PF-KEX1b, or an immunogenic composition comprising PF-KEX1b peptide or a polynucleotide encoding PF-KEX1b, in an amount effective to induce an immune response and treat or protect against disease, severe disease, or symptoms thereof, associated with infection by the Aspergillus fungal pathogen. In an embodiment, the PF-KEX1b peptide of SEQ ID NO: 1 is recombinant, recombinantly produced, and/or isolated.

In an embodiment of the methods of the above-delineated aspects, an adjuvant is administered to the subject. In an embodiment, the adjuvant is alum or ALHYDROGEL®. In an embodiment of the methods, the induced immune response treats or protects the subject against pulmonary disease or pulmonary dysfunction and/or symptoms thereof, and/or against Aspergillus-associated disease and/or symptoms thereof. In an embodiment, the Aspergillus-associated disease is aspergillosis or Invasive Pulmonary Aspergillosis (IPA) and/or symptoms thereof. In an embodiment of the methods, the subject is a mammal or a human (human patient). In an embodiment, the subject is immunocompromised or immunosuppressed. In an embodiment, the immunocompromised or immunosuppressed subject is a pre-transplant subject or a post-transplant subject. In embodiments of the methods, the immunocompromised or immunosuppressed subject is being treated for cancer, an immunodeficiency disease, a congenital disease, or an autoimmune disease. In embodiments of the method, the immunocompromised or immunosuppressed subject is being treated for an immunodeficiency disease (e.g., HIV, AIDS, HIV/AIDS), an autoimmune disease, rheumatoid arthritis, or psoriasis.

In another aspect, a vaccine comprising an effective amount of PF-KEX2b peptide as set forth in SEQ ID NO: 2 or a polynucleotide encoding PF-KEX2b is provided. In an embodiment, the PF-KEX2b peptide as set forth in SEQ ID NO: 2 is recombinant, recombinantly produced, and/or is isolated.

In another aspect, a vaccine comprising an effective amount of PF-KEX1b peptide as set forth in SEQ ID NO: 1 or a polynucleotide encoding PF-KEX1b is provided. In an embodiment, the PF-KEX1b peptide as set forth in SEQ ID NO: 1 is recombinant, recombinantly produced, and/or is isolated.

Other features and advantages of the described embodiments will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in the embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Kex1” or “Kexin” or “KEX” protein is meant a polypeptide or peptide (e.g., a fragment of the KEX polypeptide) having at least about 85% or greater amino acid identity to the amino acid sequence provided at GenBank Accession No. EU918304.1, at NCBI Accession No. XM_746441.1, at GenBank Accession No. AF022372.1, or at NCBI Accession No. XM_572303.1 and having immunogenic activity. In an embodiment, the Kex peptide is an antigenically stable active site peptide sequence. (Kutty, G. and Kovacs, J. A., 2003, Infect. Immun., 71 (1): 571-574; Lee. L. H. et al., 2000, Gene, 242 (1-2): 141-150; and Russian, D. A. et al., 1999, Proc. Assoc. Am. Physicians, 111 (4): 347-356). In an embodiment, a Kex peptide is a fragment of a naturally occurring Kexin protein or is a non-naturally occurring pan-fungal peptide or fragment thereof.

“Pan-fungal peptide 1b” (PF-KEX1b) herein refers to a KEX peptide comprising a sequence having at least 95%, at least 95.5%, at least 96%, at least 96.5%, at least 97%, at least 97.5%, at least 98%, at least 98.5%, at least 99%, at least 99.5%, or 100% identity to the amino acid sequence of Pan-fungal peptide 1b ((PF-KEX1b), (SEQ ID NO: 1), and having immunogenic activity. In some embodiments, a Pan-fungal peptide 1b (PF-KEX1b) comprises 1, 2, or 3 additional amino acids at the carboxy and/or amino terminus of the peptide, which additional amino acids do not alter the peptide's ability to stimulate an immune response in a subject. In an embodiment, the Pan-fungal peptide 1b (PF-KEX1b) herein contains the amino acid serine at positions 47 and 77 of the sequence which, for example, provides an advantageous property for manufacturing purposes and shelf-life. In an embodiment, the PF-KEX1b peptide may be beneficial in manufacturing and formulation processes. By way of nonlimiting example, the peptide is stable and is not expected to be subject to cross-linking and/or aggregation. The sequence of Pan-fungal peptide 1b (PF-KEX1b) described herein is as follows:
1 DDDGKTVDGPSPLVLRAFINGVNNGRNGLGSIYVFASGNGGIYEDNSNFD
51 GYANSVFTITIGGIDKHGKRPKYSEASSSQLAVTYAGGSA (Pan-fungal peptide 1b, (PF-KEX1b), (SEQ ID NO: 1)). In an embodiment, an immunogenically active or functional fragment of the sequence is encompassed. In some cases, the terms PF-KEX1b polypeptide and PF-KEX1b peptide are used interchangeably herein.

“Pan-fungal peptide 2b” (PF-KEX2b) herein refers to a KEX peptide comprising a sequence having at least 95%, at least 95.5%, at least 96%, at least 96.5%, at least 97%, at least 97.5%, at least 98%, at least 98.5%, at least 99%, at least 99.5%, or 100% identity to Pan-fungal peptide 2b ((PF-KEX2b), (SEQ ID NO: 2), and having immunogenic activity. In some embodiments, a Pan-fungal peptide 2 (PF-KEX2b) comprises 1, 2, or 3 additional amino acids at the carboxy and/or amino terminus, which additional amino acids do not change the peptide's immunogenicity. In an embodiment, the Pan-fungal peptide 2 (PF-KEX2b) herein includes the amino acid serine at positions 47 and 77 of the sequence which, for example, provides an advantageous property for manufacturing and formulation purposes. By way of nonlimiting example, the PF-KEX2b peptide is stable and is not expected to be subject to cross-linking and/or aggregation. The sequence of Pan-fungal peptide 2 (PF-KEX2b) described herein is as follows:
1 PDDGKTMEGPDILVLRAFINGVQNGRDGKGSIYVFASGNGGGFEDNSNFD
51 GYTNSIYSITVGAIDRKGLHPSYSEASSAQLVVTYSSGSG (Pan-fungal peptide 2, (PF-KEX2b), (SEQ ID NO: 2). In an embodiment, an immunogenically active or functional fragment of the sequence is encompassed. In some cases, the terms PF-KEX2b polypeptide and PF-KEX2b peptide are used interchangeably herein.

An exemplary Kex1 polypeptide fragment (peptide) of Pneumocystis isolated from Pneumocystis colonized non-human primates (cynomolgus macaques) and having GenBank Accession No. EU918304.1 is provided below:

| (SEQ ID NO: 3) | |
|---|---|
| 1 | DDDGKTVDGP SPLVLRAFIN GVNNGRNGLG SIYVFASGNG GIYDDNCNFD GYANSVFTIT |
| 61 | IGGIDKHGKR FAYSEACSSQ LAVTYAGGSA |

An exemplary polynucleotide sequence encoding the Kex1 polypeptide fragment (peptide) and having GenBank Accession No. EU918304.1 is provided below:

| (SEQ ID NO: 4) | |
|---|---|
| 1 | gatgacgatg gaaaaaccgt tgatgggcct tctcctcttg ttcttagagc atttattaat |
| 61 | ggagtaaata atgggaggaa tgggttgggt tctatctatg tttttgcatc aggaaatggc |
| 121 | ggaatatacg atgacaactg taattttgat ggatatgcaa atagcgtgtt tactattact |
| 181 | attggtggta tagataaaca cggaaagcgc tttgcatatt ctgaagcgtg ttcttctcag |
| 241 | ttagctgtta catatgcagg cggaagtgca |

An exemplary Kex (KexB endoprotease) polypeptide sequence of Aspergillus fumigatus (Af293) having NCBI Accession No. XM_746441.1 is provided below:

(SEQ ID NO: 5)
MRFLGSIALVLSSISVASANVRSRSYDTHEFFALHLDDSASPSHVAQLLGARHEGQIGE
LANHHTFSIPRERSSDLDALLERARAARKIRRRARDDATSQEQHNDALGGILWSQKLAP
KKRLVKRVPPPERLARTFATGKEDPVAAQSQKRIASTLGITDPIFNGQWHLFNTVQLGH
DLNVTGVWMEGITGKGVTTAVVDDGLDMYSNDLKPNYFPEGSYDFNDHTPEPRPRLSDD
KHGTRCAGEIAAARNDVCGVGVAYDSRVAGVRILSKAIDDADEATAINFAYQENDIFSC
SWGPPDDGATMEGPGILIKRAFVNGVQNGRGGKGSIFVFAAGNGASFEDNCNFDGYTNS
IYSITVGAIDREGNHPSYSESCSAQLVVAYSSGSGDAIHTTDVGTDKCYSFHGGTSAAG
PLAAGTVALALSARPELTWRDAQYLMVETAVPIHEDDGSWQVTKAGRKFSHDWGYGKVD
AYALVQKAKTWELVKPQAWFHSPWLRVQHKVPQGDQGLASSYEVTEQMMKNANIARLEH
VTVTMNVNHTRRGDLSVELRSPEGIVSHLSTTRKSDNEKAGYVDWTFMTVAHWGESGVG
RWTVIVKDTNVNEFTGEFIDWRLNLWGEAIDGANQKPHPFPDEHDDDHSIEDAIVATTS
VETGPTKTGVPGSTDDTINRPVNAKPVETQTPSPAETTATKLAPPAETRPAATATSSPT
PPAASDSFLPSFMPTFGASKRTQIWIYAAIGSIIVFCIGLGIYFQVQRRKRILNNPRDD
YDFEMIEDENALHGGNGRSGRTQRRGGELYNAFAGESDEEEPLFSDEDDEPYRDRAPSE
DRLRDTSSDDRSLRHGDH

An exemplary Kex (Kex2 proteinase) polypeptide sequence of Candida albicans having GenBank Accession No. AF022372.1 is provided below:

(SEQ ID NO: 6)
MLPIKLLIFILGYLLSPTLQQYQQIPPRDYENKNYFLVELNTTNSQKPLIDFISHYRG
HYNFEHQLSSLDNHYVFSIDKSHPHNSFLGNHNSNEYNLMKRQLGHEQDYDELISHVE
SIHLLPMKKLSKRIPVPIEMEDVVFDNRDDTGSDNHEATDEAHQKLIEIAKKLDIHDP
EFTTQWHLINLKYPGHDVNVTGLWLEDILGQGIVTALVDDGVDAESDDIKQNFNSEGS
WDFNNKGKSPLPRLFDDYHGTRCAGEIAAVKNDVCGIGVAWKSQVSGIRILSGPITSS
DEAEAMVYGLDTNDIYSCSWGPTDNGKVLSEPDVIVKKAMIKGIQEGRDKKGAIYVFA
SGNGGRFGDSCNFDGYTNSIYSITVGAIDYKGLHPQYSEACSAVMVVTYSSGSGEHIH
TTDIKKKCSATHGGTSAAAPLASGIYSLILSANPNLTWRDVQYISVLSATPINEEDGN
YQTTALNRKYSHKYGYGKTDAYKMVHFAKTWVNVKPQAWYYSDIIEVNQTITTTPEQK
APSKRDSPQKIIHSSVNVSEKDLKIMNVERVEHITVKVNIDSTYRGRVGMRIISPTGV
ISDLATFRVNDASTRGFQNWTFMSVAHWGETGIGEWKVEVFVDDSKGDQVEINFKDWQ
FRIFGESIDGDKAEVYDITKDYAAIRRELLEKEKQNSKSTTTTSSTTTATTTSGGEGD
QKTTTSAENKESTTKVDNSASITTSQTASLTSSNEQHQPTESNSDSDSDTDDENKQEG
EEDNDNDNDNGNKKANSDNTGFYLMSIAVVGFIAVLLVMKFHKTPGSGRRRRRRDGYE
FDIIPGEDYSDSDDDEDDSDTRRADDDSFDLGHRNDQRVVSASQQQRQYDRQQDEARD
RLFDDFNAESLPDYENDMFKIGDEEEEEEEEEEGQQSAKAPSNSEGNSGTSTKK

An exemplary Kex polypeptide sequence of Cryptococcus neoformans (JEC21) having NCBI Accession No. XM_572303.1 is provided below:

(SEQ ID NO: 7)
MRTLLSLWGILLALIVPPSLALQRPQPRSYDTHAYYALELDPSISPAAALQLSKSLGV
ELVERIGELDGHWLVRTEGWTPEHASITKRSVSHDPILKRWEALPSSLGKKSLTPLSL
KQRAKRHKSYSPRSRHSRDDRTELLYAQNELHLADPMLDQQWHLINTQMKDIELNVTG
LWGRGITGEGVHVVIIDDGLDVESKDLKDNFFAEGSYDFNDHTELPIPRLKDDQHGTR
CAGEIAAVPNDVCGVGVAYDSKIAGVRILSAPISDADEAAALNYAYQLNDIYSCSWGP
PDDGRSMEAPDGLILKAMVNGVQKGRDGKGSVFVFAAGNGGGSDDQCNFDGYTNSIFS
VTVGAVDRKGLHPYYSEMCAAMMVVAPSSGSGDHIHTTDVGKDKCSHSHGGTSAAAPL
AVGVFALALSVRPDLTWRDIQHLAVRHAVFFNPDDPAWELTAAGRHFSYKYGYGKLDA
GLFVEAAEKWQLVKPQTWYDSPSVYLPTTSPADVTRRQDEAADGPTSSDEETSNPPPV
VEPSGSFITEDGVISTYEVTQSMLFDANFERLEHVTVRVWIDHQRRGDVEVELTSPNG
VVSVLCRQRRFDNADSGFPGWKFMSLKHWDENPVGTWTIKVKDQVNPDKTGRFVAWSL
QLWGESVDPALAKLWAPAEEGQPDEEQTGSNPSTTVSQKPKPTALLPGDHGEASGEAT
QPGLGSATAHPQPTSTTGDAGNVAEPTGPTDADADEGFFSGISNLASSSTWLAGAGAI
IILSGAAIGAFFFIRARRQKRNLFGLSNNGQGARGAYEPVDDVQMSLLERGRRKFGKS
KSESQGTKDLYDAFGDGPSDEEEEDLDERTALRYHDGFLEDDEPNEVGPKTEYKDEPE
SEPETFKDGEETVGTKDKGKGKGPSEGESGSGSSSSWQDAADEEARV

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies of the described embodiments may exist in a variety of forms including, for example, polyclonal antibodies, monoclonal antibodies (Mabs), Fv, Fab and F(ab′)₂, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody. An antibody fragment can refer to the antigenic determining variable regions (e.g., heavy and light chain variable regions) of an intact antibody. Examples of antibody fragments include, but are not limited to, Fab, Fab′, F(ab′)₂, and Fv fragments, linear antibodies, scFv antibodies, single-domain antibodies, such as camelid antibodies (Riechmann, 1999, Journal of Immunological Methods, 231:25-38), composed of either a V_{L} or a V_{H} domain which exhibit sufficient affinity for the target, and multispecific antibodies formed from antibody fragments. An antibody fragment may be derived from a chimeric antibody, a human antibody, a humanized antibody, or a portion of a human antibody or a humanized antibody.

Antibodies can be made by any of the methods known in the art utilizing a polypeptide (e.g., a Kexin polypeptide), or immunogenic peptide fragments thereof, as an immunogen. One method of obtaining antibodies is to immunize suitable host animals with an immunogen and to follow standard procedures for polyclonal or monoclonal antibody production. The immunogen facilitates the presentation of the immunogenic fragments on the cell surface. Immunization of a suitable host can be carried out in a number of ways. Nucleic acid sequences encoding a polypeptide as described herein, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. To produce large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

By “anti-Kexin antibody,” “anti-Kex antibody,” or “anti-Kex1 antibody” is meant an antibody or an antigen binding fragment thereof that selectively binds to a Kexin polypeptide or peptide, including, for example, a Kex1 peptide fragment of a fungal pathogen, such as Candida albicans, Pneumocystis hominis, Pneumocystis jirovecii (aka carinii), Aspergillus fumigatus and Cryptococcus neoformans as described herein, or to PF-KEX1b or PF-KEX2b as described herein. In various embodiments, anti-Kexin antibody or anti-Kex1 antibody specifically binds a binding site of a Kexin protein or peptide. In specific embodiments, the anti-Kexin antibody or anti-Kex1 antibody specifically binds a binding site of a Kexin protein or peptide of a fungal pathogen, for example, one or more of a Candida, Pneumocystis, Aspergillus and/or Cryptococcus fungal pathogen. In an embodiment, the antibody binds to PF-KEX1b. In an embodiment, the antibody binds to PF-KEX2b.

An “antiserum” refers to blood serum that contains one or more antibodies directed against a specific antigen. Antiserum containing antibodies may be obtained from the blood or serum of an animal (a mammal), including a human, that has been immunized or inoculated with an immunogen (or an antigen material) either by injection, typically into the bloodstream or tissues, or by infection. In an embodiment, the animal (a mammal), including a human, may be immunized or inoculated with the blood or serum of an organism or individual whose immune system has been stimulated to generate an immune response (e.g., antibody production) by infection or natural contact with an antigenic material or immunogen. In this case, an antiserum contains anti-Kex peptide antibodies, e.g., polyclonal antibodies or populations of monoclonal antibodies, generated or produced by an immunized, inoculated, or exposed donor subject against a Kex peptide immunogen, or a polynucleotide encoding the Kex peptide immunogen, derived from a fungal pathogen, e.g., Pneumocystis (e.g., Pneumocystis jirovecii), for example a PF-KEX1b or a PF-KEX2b peptide immunogen. Such antiserum, isolated (and/or purified) from the donor subject is used to immunize (i.e., administer to) another (unrelated) subject to provide immunity (acquired immunity) against infection or disease caused by or associated not only with the Pneumocystis pathogen as original source of the immunogen, but also with other fungal pathogens that have a Kex peptide that is also targeted and recognized by the antibodies in the antiserum. In embodiments, the fungal pathogens include Pneumocystis species (spp.) and one or more of Candida spp. or Candida albicans, Aspergillus spp. or Aspergillus fumigatus, or Cryptococcus spp. or Cryptococcus neoformans. In this way, a subject who receives the antiserum, i.e., antibodies in the antiserum, is treated or protected against infection and/or disease caused by more than one fungal pathogen. Such antiserum-derived immunoprotection against multiple fungal pathogens constitutes an acquired or passive immunity obtained by the recipient subject and imparted from the donor subject's isolated antiserum. As will be appreciated by one skilled in the art, blood serum is the amber-colored, protein-rich liquid component of blood that separates from the clot when blood coagulates. The serum component containing one or more antibodies (cross-protective antibodies) is termed “antiserum.” In an embodiment, the antiserum is an isolated antiserum, e.g., isolated from a donor subject. In an embodiment, an isolated antiserum may be processed by methods used by one skilled in the art, such as dilution, concentration (e.g., via filtration or centrifugation or both), chromatography, purification to remove ions or extraneous protein, and the like, prior to its use as a treatment or protective therapeutic as described herein. In an embodiment, an isolated antiserum may be further purified after isolation. In an embodiment, an isolated antiserum is not further processed or purified. In an embodiment, antibodies, or antigen-binding fragments thereof, contained in an isolated antiserum may be further isolated by methods practiced by those having skill in the art, such as, without limitation, by affinity chromatography, size exclusion chromatography, immunoprecipitation, dialysis, HPLC chromatography, etc.

By “biological sample” is meant any liquid, cell, or tissue obtained from a subject. In some embodiments, the biological sample is blood, serum, plasma, cerebrospinal fluid, bronchoalveolar lavage, sputum, tears, saliva, urine, semen, stool, etc. In an embodiment, a tissue preparation is encompassed, in which tissue is homogenized or otherwise prepared to generate a suspension containing cells.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte that is detected or that is to be detected.

By “disease” is meant any condition, dysfunction, or disorder that damages or interferes with the normal function of a cell, tissue, or organ. In various embodiments, non-limiting examples of diseases, or symptoms thereof, associated with infection by fungal pathogens include pulmonary (lung) disease, brain disease, e.g., meningitis. Non-limiting examples of pulmonary diseases include Chronic Obstructive Pulmonary Disease (COPD), which is a progressive lung disease that includes emphysema, chronic bronchitis, refractory (non-reversible) asthma, pneumonia (e.g., Pneumocystis pneumonia) and some forms of bronchiectasis. Non-limiting examples of diseases associated with infection by Aspergillus spp. include aspergillosis or Invasive Pulmonary Aspergillosis (IPA) and/or symptoms thereof. Non-limiting examples of diseases associated with infection by Candida spp., e.g., C. albicans, C. aurus, C. glabrata, C. tropicalis, C. parapsilosis, or C. krusei may include, without limitation, vaginitis and infections of the urinary tract (UTIs). Non-limiting examples of diseases, conditions, pathologies, or symptoms thereof, associated with subjects (patients) who are immunosuppressed or immunocompromised include organ or tissue transplant or post-transplant, cancer, an immunodeficiency disease (e.g., HIV, AIDS, HIV/AIDS), a congenital disease, or an autoimmune disease, rheumatoid arthritis, or psoriasis, and the like. In an embodiment, the term disease embraces an immunocompromised subject or patient, or an immunosuppressed subject or patient. In an embodiment, a subject may be at high risk of, or susceptible to, fungal infections and associated diseases as a consequence of being immunocompromised or immunosuppressed. In embodiments, the PF-KEX1b and PF-KEX2b peptide immunogens, and/or antibodies or antisera generated against these peptide immunogens, may be of prophylactic and/or therapeutic use in subjects having, or at risk of having, inflammation-related diseases, diabetes (Type 1 or Type 2 diabetes), asthma, severe asthma, COPD, and immune or autoimmune disorders, such as HIV, AIDS, HIV/AIDS, including symptoms thereof.

By “effective amount” is meant the amount of a required to ameliorate the symptoms of a disease relative to an untreated patient. The effective amount of active compound(s) used to practice the methods as described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. An immunologically effective amount of an isolated antiserum as described herein is an amount required to treat a fungal infection or disease associated with one or more of the fungal pathogens described herein. By way of example, an effective amount of an isolated antiserum may be determined by measuring the amount or titer of antibodies directed against the desired immunogen present in the serum by methods known and practiced in the art. The range of typical dosages for passive immunotherapy (i.e., the administration of antiserum containing antibodies) includes about 0.3 mg to about 100 mg/kg of total body weight. Following passive immunotherapy, treatment efficacy is typically conducted, as individual patients respond differently to therapies. Adjustment of the dosage may be modified as needed. Treatment regimens can be determined by methods known and practiced by those having skill in the art. In one embodiment, the amount is sufficient to induce an immune response.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In embodiments, the fragment or portion retains activity or function, such as, without limitation, antigen binding function or immunogenicity.

By “genetic vaccine” is meant an immunogenic composition comprising a polynucleotide encoding an antigen.

By “immune response” is meant is any activity of the immune system that is generated against an antigen. In some embodiments, the immune response is an innate or an adaptive immune response that protects a subject from infection with a pathogen (e.g., fungal pathogen) or treats a pathogen infection. In some embodiments, an immune response involves the generation of antibodies against an antigen. In some embodiments, an immune response encompasses a B cell response, a T cell response, or both a B cell and a T cell response.

The term “immunocompromised” refers to a subject having a weakened or impaired immune system and/or associated immune response to a pathogen, pathogenic antigen, disease, etc. A subject may be immunocompromised as a consequence of taking immunosuppressive drugs, or by being afflicted with a disease or pathology that affects the subject's immune system, such as certain congenital diseases. The term “immunosuppressed” refers to a subject whose immune system and associated immune response to pathogens, pathogenic antigens, disease, etc. is partially or completely suppressed, for example, by a reduction in the activity or efficiency in the immune system. Immunosuppression of a subject's immune system or immune response may occur naturally due to a disease or disorder in the subject, or may be induced in the subject by the administration of immunosuppressive agents, drugs, e.g., anti-cancer drugs, compounds, and the like. In some cases, a subject who is immunosuppressed or is undergoing immunosuppression, or who has a weakened immune system due to a disease or condition (e.g., chemotherapy or an immune deficiency disease) is said to be immunocompromised.

By “immunogenic composition” is meant a composition comprising an antigen or immunogen or a polynucleotide encoding the antigen or immunogen, wherein the composition elicits an immune response in an immunized subject.

The terms “isolated,” “purified,” or “biologically pure” refer to material that is free to varying degrees from components which normally accompany it as found in its native state or environment. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” or “isolated peptide” is meant a polypeptide or peptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide or peptide as described herein. An isolated polypeptide or peptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, isolating, purifying, or otherwise acquiring the agent.

By “opportunistic infection” is meant an infection caused by pathogens such as fungal pathogens, bacteria, viruses, protozoa, or parasites that take advantage of an opportunity to infect a subject (host) that is not normally available, for example, a host having a weakened immune system, an immunocompromised host, an immunosuppressed host, a host with altered microbiota or microflora, or a host having protective integumentary barriers that have been damaged or breached. In an embodiment, an opportunistic infection is caused by one or more fungal pathogens as described herein.

By “reduces” or “diminishes’ is meant a negative alteration of at least 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody or antigen binding fragment thereof that recognizes and binds a polypeptide or peptide, but which does not substantially recognize and bind other molecules in a sample, for example, a biological sample, which naturally includes a polypeptide or peptide as described herein. Cross-reactive binding includes specific binding (e.g., by an antibody or an antigen binding fragment thereof) to an original polypeptide or peptide antigen/immunogen as well as binding to a polypeptide or peptide other than the original antigen/immunogen.

Nucleic acid molecules useful in generating a recombinant immunogen or a vaccine include any nucleic acid molecule that encodes a polypeptide or a peptide fragment thereof, such as a Kex wildtype or pan-fungal polypeptide or Kex wildtype or pan-fungal peptide described herein. Such nucleic acid molecules need not be 100% identical with an endogenous nucleic acid sequence, but will typically exhibit substantial identity to an endogenous sequence. Polynucleotides having “substantial identity” to an endogenous sequence are typically capable of hybridizing with at least one strand of a double-stranded nucleic acid molecule. Nucleic acid molecules may include any nucleic acid molecule that encodes a polypeptide or a peptide fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, or a murine, bovine, equine, canine, ovine, or feline mammal. In an embodiment, the subject is a human. In an embodiment, a subject is a human patient who is undergoing treatment for infection or disease caused by one or more pathogenic fungi, such as Pneumocystis, Aspergillus, Candida, or Cryptococcus. In an embodiment as subject is a human patient who is at risk of infection (e.g., opportunistic infection) or disease caused by one or more pathogenic fungi, such as Pneumocystis, Aspergillus, Candida, or Cryptococcus. In an embodiment, a subject is a mammalian (e.g., a human; a non-human primate) donor subject from whom antiserum containing anti-fungal Kex peptide antibodies is obtained or isolated. In an embodiment, a subject is a mammalian (e.g., a human; a non-human primate) recipient subject who receives an isolated antiserum and acquires protective immunity (and treatment) against multiple fungal pathogens.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. One skilled in the art will recognize that depending on the expression vector used, additional nucleotides may need to be added to the 5′ end of the foreign nucleic acid to be inserted into the vector to maintain the proper reading frame.

By “vaccine” is meant a preparation of immunogenic material (e.g., protein or nucleic acid; particles) capable of stimulating (eliciting) an immune response, administered to a subject to treat a disease, condition, or pathology, or to prevent a disease, condition, or pathology, such as an infectious disease (caused by fungal infection, for example). The immunogenic material may include, for example, attenuated or killed microorganisms (such as attenuated viruses), or antigenic proteins, peptides or DNA derived from such microorganisms. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous or intramuscular. Vaccines may also be administered with an adjuvant to boost the immune response.

Ranges provided herein are understood to be shorthand for all the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, abating, diminishing, or ameliorating a disease, disorder and/or symptoms associated therewith. It will be appreciated that, although not precluded, treating a disease, disorder and/or symptoms associated therewith does not require that the disease, disorder, condition or symptoms associated therewith be eliminated.

As used herein, a therapeutic that “prevents” a disorder or condition refers to a compound or material that, in a statistical sample, reduces the occurrence of the disorder or condition in the treated sample relative to an untreated control sample, or delays the onset or reduces the severity of one or more symptoms of the disorder or condition relative to an untreated control sample. In an embodiment, a preventive therapeutic is an antibody or an antigen binding fragment thereof. In a particular embodiment, a preventive therapeutic is an isolated antiserum containing anti-Kex peptide antibodies or antigen binding fragments thereof as described herein.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

Exemplary embodiments as described herein are provided below as drawings and figures related to the described embodiments in their various and nonlimiting aspects.

FIG. 1 illustrates a graph showing that the PF.KEX2b immunogen generated an antibody response and the production of antibodies as measured by antibody titer in plasma of animals (mice) that had received the PF-KEX2b immunogen. The results present the mean plasma anti-PF.KEX2b-specific immunoglobulin G (IgG antibody) reciprocal endpoint titer (RET), as determined by enzyme-linked immunosorbent assay (ELISA), following three immunizations of the mice with either 10 μg (represented by “x” markings on graph) or 20 μg (represented by solid black circles on graph) of the PF.KEX2b immunogen and alum (ALHYDROGEL®; InvivoGen, San Diego, CA) adjuvant. Time points of immunization of the animals are indicated by arrows on the graph. “Wpv”: weeks post-vaccination.

FIGS. 2A and 2B present a study design diagram and a graph showing survival curves related to a fungal challenge study of invasive pulmonary aspergillosis in an immunosuppression mouse model. FIG. 2A presents the design of the fungal challenge study in animals. FIG. 2B shows a survival curve of animals immunized with the PF.KEX2b peptide immunogen (represented by “x” markings on graph), compared to sham-immunized control animals. The PF.KEX2b-immunized animals were significantly protected from developing aspergillosis disease following immunosuppression (elicited using tacrolimus and hydrocortisone), compared to the sham-immunized controls (*p=0.0323, by Mantel-Cox test). The results demonstrated that immunosuppressed animals immunized with the PF.KEX2b peptide immunogen in conjunction with an adjuvant (ALHYDROGEL®) had a significant reduction in mortality related to Aspergillus fungal infection compared to sham-immunized control animals (p=0.0323).

FIGS. 3A and 3B show Coomassie stained gels and western blot analyses of PF-KEX2a peptide (i.e., Pan-fungal peptide 2; see, e.g., US Pub. No. 2022/0184190, the contents of which are incorporated by reference herein) and the PF-KEX2b peptide described herein. FIG. 3A: PF-KEX2a protein quality analysis prior to use in immunization/vaccination of animals and following prolonged storage at −80 C. Following overnight dialysis of the PF-KEX2a peptide in PBS, the dialyzed protein was frozen in several aliquots for in vivo use at later timepoints. The quality of the protein was assessed by Coomassie staining following gel electrophoresis and PF-KEX2a immunoblot analysis immediately following dialysis (left panel), after storage at −80° C. for 47 days (center panels), and after storage for 90 days (right panels). The asterisk (*) indicates the post-dialysis pool that was aliquoted and stored at −80° C. for subsequent use for immunization/vaccination 47 and 90 days later. The PF-KEX2a peptide runs on the gel at a similar molecular weight in the presence or absence of β-mercaptoethanol (left panels) and does not degrade over time. The concentration of each aliquot following dialysis, after storage for 47 days, and after storage for 90 days at 80° C. was 1.23 mg/ml, 1.20 mg/ml, and 1.28 mg/ml, respectively, when measured by Bradford assay. The PF-KEX1b and PF-KEX2b peptides described herein are expected to exhibit similar protein quality analysis results. FIG. 3B: PF-KEX2b protein quality analysis prior to immunization/vaccination of animals and storage at −80° C. Following overnight dialysis of the PF-KEX2b peptide in PBS, the dialyzed protein was frozen in several aliquots for in vivo use at later timepoints. The quality of the protein was assessed by Coomassie staining following gel electrophoresis (left panel) and PF-KEX2b immunoblot analysis (right panel) following dialysis and storage at −80° C.

### DETAILED DESCRIPTION OF THE EMBODIMENTS

Featured herein are compositions and methods for treating or preventing infection by one or more fungal pathogens, or disease associated with infection by more than one fungal pathogen, in which the pathogens are of different etiologies. By way of specific example, disease-causing fungal pathogens include Aspergillus, a common mold, which causes aspergillosis, allergic reactions, lung infections and other health problems; Pneumocystis, which colonizes lung tissue and causes severe pneumonia after infection; Candida, which typically reside in the intestinal tract and mucous membranes and can cause thrush, infections and invasive candidiasis upon systemic infection, especially in those in poor health or with weak immune systems; and Cryptococcus, which can infect the lungs, where it can cause pneumonia-like illness, and the brain, where it can cause meningitis. In an embodiment, the compositions and methods are useful for treating or preventing disease, e.g., aspergillosis, associated with infection by Aspergillus fungal pathogen.

The described embodiments are based, at least in part, on the discovery that mammalian subjects, immunized with a non-naturally occurring polypeptide or peptide (referred to herein as a pan-fungal (PF) peptide) having sequence similarity to the Kexin (Kex or Kex1) protein, e.g., Pan-fungal peptide 1b (called “PF-KEX1b” herein), (SEQ ID NO: 1), and Pan-fungal peptide 2 (called “PF-KEX2b” herein), (SEQ ID NO: 2) described herein, generate antibodies that specifically bind Kex peptides of distinct fungal pathogens, including Pneumocystis, Candida, Aspergillus, and Cryptococcus. Accordingly, the pan-fungal peptides as described herein are useful for treating or preventing diseases caused by or associated with infection by any one or more of Pneumocystis, Candida, Aspergillus and Cryptococcus fungal pathogens. In an embodiment, the PF-KEX1b and PF-KEX2b peptides described herein are useful for treating or preventing disease, e.g. aspergillosis, caused by or associated with infection by the Aspergillus fungal pathogen. The PF-KEX1b and PF-KEX2b peptides, which are stable peptides, may also provide beneficial properties, e.g., a lack or reduction of cross-linking and/or aggregation, which are useful for manufacturing or formulating the peptides, for example, for commercial production and use.

Antibody-containing antiserum generated in response to administration of the non-naturally occurring pan-fungal Kex peptides PF-KEX1b and/or PF-KEX2b, or a polynucleotide encoding PF-KEX1b or PF-KEX2b, can also serve as a treatment for disease caused by infection by one or more fungal pathogens and can provide immunity against one or more fungal pathogens in a subject or in another or unrelated subject (i.e., a recipient subject) who receives the antiserum via a suitable mode and route of administration. It will be appreciated by the skilled practitioner that, as used herein, a subject from whom an antiserum is obtained or isolated is a “donor subject,” and a subject to whom the isolated antiserum is administered or provided is a “recipient subject.” In embodiments, a subject is a mammal, particularly a human being or a non-human primate. A recipient subject may be a patient or an individual in need of treatment for or protection from disease caused by infection by one or more of the Pneumocystis, Candida, Aspergillus and/or Cryptococcus fungal pathogens. In an embodiment, the fungal pathogen is Aspergillus.

The production of such immunologically cross reactive antisera (and antibodies therein) produced in subjects (e.g., donor subjects) immunized with, or exposed to, PF-KEX1b or PF-KEX2b, or a polynucleotide encoding the pan-fungal Kex peptide, that react with the Kex peptides of multiple fungal pathogens, such as Candida, Aspergillus and Cryptococcus fungal pathogens, is surprising and unexpected, particularly in view of the low amount of amino acid sequence identity (about 48%-70% variability) among the Kex peptides of the Pneumocystis, Candida, Aspergillus and Cryptococcus fungal pathogens, and in view of the overall amount of variability in the amino acid sequences (ranging from about 70% to 96% variability) between the Kex1 peptide of Pneumocystis and the Kex peptides of the Candida, Aspergillus and Cryptococcus fungal organisms.

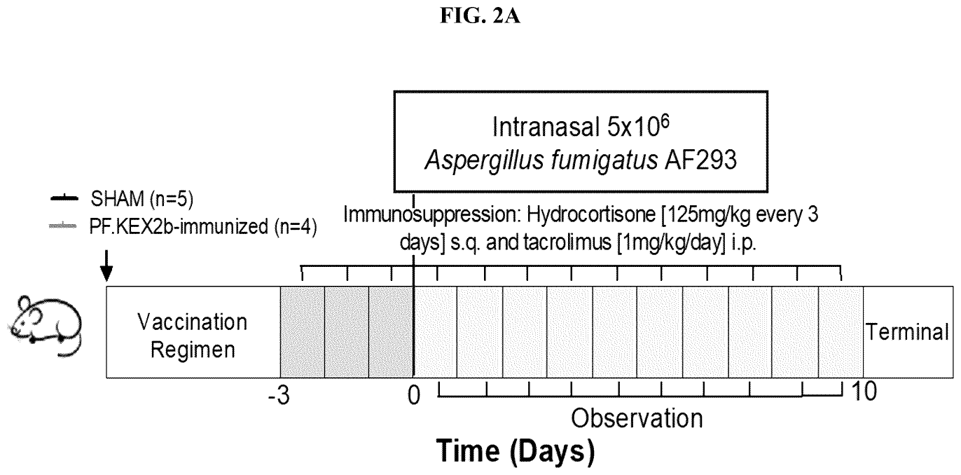

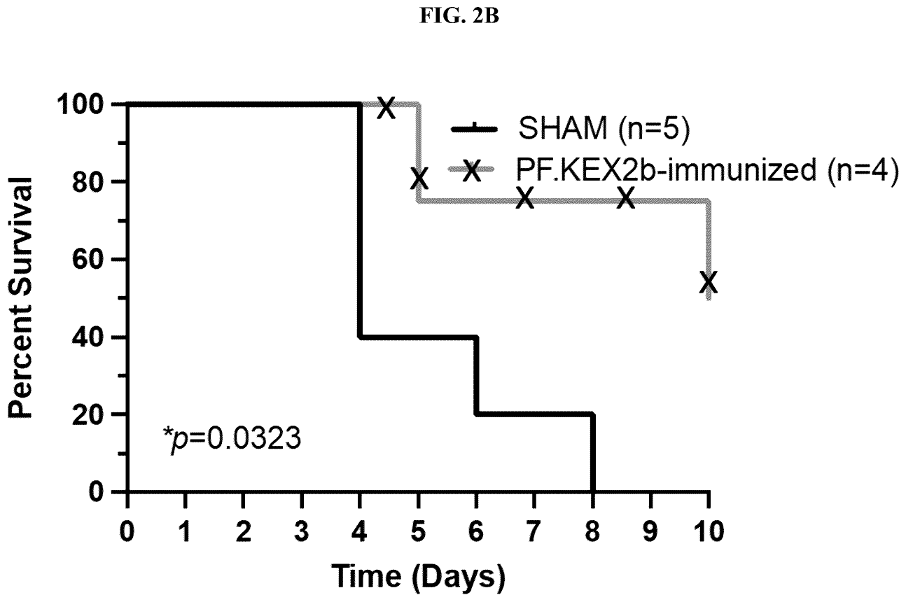

Also embraced herein is an immunogenic composition comprising PF-KEX1b or PF-KEX2b, or a polynucleotide encoding the pan-fungal peptide, that elicits a potent immune response in a subject following administration of the composition and the production of antiserum in the subject that contains antibodies or antigen binding fragments thereof that bind to (react with) not only the immunizing PF-KEX1b or PF-KEX2b peptide immunogen, but also a similar, but nonidentical, kexin antigen produced by other fungal pathogens. In an embodiment, the PF-KEX2b peptide, or a composition thereof, used as an immunogen administered to a subject generates an immune response in the subject that treats disease (e.g., aspergillosis) and/or reduces mortality associated with infection by the Aspergillus (e.g., Aspergillus fumigatus) fungal pathogen. See, e.g., FIGS. 2A and 2B. In an embodiment, the subject is immunosuppressed or immunocompromised.

One benefit of the described methods is the provision of treatment or prevention of disease and the symptoms and adverse effects thereof associated with infection by one or more different fungal pathogens using only one therapeutic agent, i.e., a composition comprising the non-naturally occurring PF-KEX1b or PF-KEX2b peptide, or a polynucleotide encoding the pan-fungal Kex peptide, or an antiserum (isolated antiserum) generated in response to administration of the non-naturally occurring PF-KEX1b or PF-KEX2b peptide, or polynucleotide encoding the pan-fungal Kex peptide, that treats and/or cross-protects against multiple fungal organisms and treats or prevents diseases and symptoms thereof, for example, pulmonary disease and poor pulmonary performance, associated with infection (and colonization) by the different fungal pathogens, e.g., one or more, or at least two or more fungal pathogens. In an embodiment, the different fungal pathogens include Pneumocystis, Aspergillus, Candida, and Cryptococcus, particularly, one or more, or at least two or more thereof. In one embodiment, a composition is provided that comprises the non-naturally occurring PF-KEX1b or PF-KEX2b peptide, or a polynucleotide encoding the pan-fungal Kex peptide, that elicits the production of antibodies that specifically react with a Kex peptide derived from one or more of Pneumocystis (Pneumocystis jirovecii), Aspergillus (Aspergillus fumigatus), Candida (Candida albicans), or Cryptococcus (Cryptococcus neoformans). Another embodiment provides an antiserum produced in a subject immunized with non-naturally occurring PF-KEX1b or PF-KEX2b peptide, or a polynucleotide encoding the pan-fungal Kex peptide, that contains antibodies that also specifically react with a Kex peptide derived from one or more of Pneumocystis (Pneumocystis jirovecii), Aspergillus (Aspergillus fumigatus), Candida (Candida albicans), or Cryptococcus (Cryptococcus neoformans). Accordingly, the antiserum is cross-protective (e.g., cross-reactive with Kex peptides of multiple fungal types) and affords treatment and/or protection against diseases as described herein, including diseases associated with infection by one or more fungal organisms when provided to another (e.g., unrelated) subject in need thereof. In an embodiment, the antiserum is an isolated antiserum. In an embodiment, the isolated antiserum is administered in a pharmaceutically acceptable composition.

The methods and compositions comprising the PF-KEX1b or the PF-KEX2b peptide, described herein offer economic, medical and practical benefits in the treatment and prevention of fungal disease, such as pulmonary disease, or types of brain infections, associated with infection and colonization by different types of fungal pathogens. In an embodiment, the methods and compositions may comprises the PF-KEX1b and the PF-KEX2b peptide immunogens.

### Therapeutic Methods

The methods and compositions provided herein can be used to treat or prevent disease associated with or caused by one or more of the fungal pathogens Pneumocystis, Aspergillus, Candida, and Cryptococcus, and, in particular, Pneumocystis hominis or jirovecii, Aspergillus fumigatus, Candida albicans, or Cryptococcus neoformans. The methods and compositions provided herein can provide immune protection in a subject against serious or severe disease and the symptoms thereof caused by at least one, and particularly more than one, of these fungal organisms following infection. The methods and compositions provided herein can immunize a recipient subject against contracting serious infection and/or disease caused by at least one and particularly by more than one of these fungal organisms. In general, an immunogenic composition comprising a non-naturally occurring PF-KEX1b or the PF-KEX2b peptide, a polynucleotide encoding the pan-fungal Kex peptide, or an isolated antiserum containing one or more antibodies generated against the non-naturally occurring PF-KEX1b or the PF-KEX2b peptide can be administered therapeutically and/or prophylactically to provide immunity against other pathogenic fungal organisms that express a Kex protein or peptide antigen. The methods include administering an immunologically effective amount of the immunogenic composition comprising PF-KEX1b or PF-KEX2b, a polynucleotide encoding the pan-fungal Kex peptide, an isolated antiserum, or immune serum or immune plasma as described herein to an individual, alone, or in a physiologically acceptable carrier, excipient, or diluent. In an embodiment, the immunogenic composition comprising PF-KEX1b or PF-KEX2b, a polynucleotide encoding the pan-fungal Kex peptide, or isolated antiserum is in a pharmaceutically acceptable composition.

Provided and described herein are methods of treating or preventing disease or the severity of effects of infection by one or more fungal pathogens (e.g., one or more of Pneumocystis, Aspergillus, Candida, or Cryptococcus, more specifically, Pneumocystis hominis or jirovecii, Aspergillus fumigatus, Candida albicans, or Cryptococcus neoformans fungi), and/or diseases, disorders, or symptoms thereof, which comprise administering a therapeutically effective amount of an immunogenic composition comprising the non-naturally occurring PF-KEX1b or PF-KEX2b peptide, a polynucleotide encoding the pan-fungal Kex peptide, or an isolated antiserum generated in response to immunization with the non-naturally occurring PF-KEX1b or PF-KEX2b peptide as described herein in a recipient subject (e.g., a mammal such as a human patient) in need thereof. In an embodiment, the antiserum contains antibodies that specifically target Kexin protein or Kex peptide to neutralize the activity of Kex proteinase. In an embodiment, the isolated antiserum containing antibodies directed against the PF-KEX1b or the PF-KEX2b peptide allows the recipient subject to achieve and passively acquire protective immunity and/or treatment against multiple fungal pathogens.

In an embodiment, a method as described herein involves treating a subject suffering from or susceptible to an infection by Pneumocystis, Aspergillus, Candida, or Cryptococcus, or disease or symptom thereof (e.g., pulmonary disease, COPD, aspergillosis, etc.) associated with or caused by one or more of these fungal pathogens. The method includes the step of administering to the subject (e.g., a mammal or human patient) the PF-KEX1b or the PF-KEX2b peptide, a polynucleotide encoding the PF-KEX1b or the PF-KEX2b peptide, an immunogenic composition comprising the PF-KEX1b or the PF-KEX2b peptide peptide or encoding polynucleotide, or an isolated antiserum generated against the PF-KEX1b or the PF-KEX2b peptide in an amount that is sufficient to treat an infection, disease, disorder, or symptom thereof, caused by one or more different types of fungal organisms under conditions such that the infection, disease, disorder, or symptom thereof, is treated. In an embodiment, the isolated antiserum is in a pharmaceutically acceptable composition.

Also provided and described are methods of treating or preventing disease, serious or severe disease, and/or the symptoms thereof, caused by or associated with infection by one or more than one type of fungal pathogen, in which the methods comprise administering a therapeutically effective amount of an immunogenic composition comprising a non-naturally occurring PF-KEX1b or PF-KEX2b peptide described herein, a polynucleotide encoding the pan-fungal Kex peptide, or an isolated antiserum (e.g., comprising one or more antibodies or an antigen binding fragment thereof) generated in response to the non-naturally occurring PF-KEX1b peptide or PF-KEX2b peptide immunogen administered to a subject (e.g., a mammal such as a human). In various embodiments, the method prevents disease, severe disease, and/or mortality associated with infection by one or more fungal pathogens selected from Pneumocystis, Aspergillus, Candida, or Cryptococcus in a subject susceptible to fungal infection, disease, severe disease, or symptoms thereof (e.g., COPD, lung/pulmonary disease, poor pulmonary function, aspegillosis, and the like). In an embodiment, the method includes the step of administering to a recipient mammal a therapeutic amount of an immunogenic composition comprising a non-naturally occurring PF-KEX1b or PF-KEX2b peptide, a polynucleotide encoding the pan-fungal Kex peptide, or an isolated antiserum generated in response to immunization of a subject with the non-naturally occurring PF-KEX1b or PF-KEX2b peptide in an amount sufficient to prevent or treat the disease, disorder, or symptom thereof, (and severe forms thereof) and under conditions such that the disease disorder, or symptom thereof is treated. In an embodiment, the method includes the step of administering to a recipient mammal a therapeutic, prophylactic, or preventive amount of an immunogenic composition comprising a non-naturally occurring PF-KEX1b or PF-KEX2b peptide, a polynucleotide encoding the pan-fungal Kex peptide, or an antiserum generated in response to immunization of a subject with the non-naturally occurring PF-KEX1b or PF-KEX2b peptide in an amount sufficient to treat or prevent the infection, disease, disorder, or symptom thereof, under conditions such that the infection, disease disorder, or symptom thereof is treated or prevented. In an embodiment, the isolated antiserum is in a pharmaceutically acceptable composition. In an embodiment, the recipient mammal is a human patient in need of treatment.

Treatment will be suitably administered to subjects, particularly humans, suffering from, having, susceptible to, or at risk for, infection by, one or more than one fungal organism, in particular, one or more than one of Pneumocystis, Aspergillus, Candida, or Cryptococcus, and in particular, Pneumocystis hominis or jirovecii, Aspergillus fumigatus, Candida albicans, or Cryptococcus neoformans, or a disease, pathogenic condition, or symptom thereof associated with infection by one or more of the fungal pathogens. Determination of those subjects who are “at risk” can be made by any objective or subjective determination by a diagnostic test or opinion of a subject or health care provider (e.g., genetic test, enzyme test or assay, or protein marker (such as levels of anti-Kex antibodies, e.g., in serum), family history, and the like). The methods herein also include administering to the recipient subject (including a subject identified as in need of such treatment or as being at risk of infection) an effective amount of an anti-fungal pathogen immunogenic composition comprising a non-naturally occurring PF-KEX1b or PF-KEX2b peptide, a polynucleotide encoding the pan-fungal Kex peptide, or an antiserum generated in response to immunization of a subject with the non-naturally occurring PF-KEX1b or PF-KEX2b peptide and isolated from the subject as described herein. Identifying a subject in need of such treatment can involve the judgment of the recipient subject or a health care or medical professional and can be subjective (e.g., opinion) or objective (e.g., measurable by a test or diagnostic method). In an embodiment, the isolated antiserum is provided in a pharmaceutically acceptable composition.

In some aspects, methods of treating or preventing a fungal pathogen-associated disease or condition (e.g., pulmonary infection, pulmonary disease or disorder, pneumonia, COPD, aspergillosis, and the like) in a subject are featured, in which the methods involve administering to a subject in need thereof an effective amount of an immunogenic composition comprising a non-naturally occurring PF-KEX1b or PF-KEX2b peptide described herein, a polynucleotide encoding the pan-fungal Kex peptide, or an isolated antiserum generated in an individual in response to administration of the non-naturally occurring PF-KEX1b or PF-KEX2b peptide and obtained from the individual who has produced an antibody immune response against the PF-KEX1b or PF-KEX2b peptide, such that the subject is therapeutically and/or prophylactically treated against disease, or symptoms thereof, associated with a fungal pathogen.

Provided in another aspect are methods of treating or preventing disease and/or symptoms thereof associated with fungal infection in a patient who is receiving or who has received immune suppressive drugs or medication and who, as a result of drug-induced immune system suppression, is susceptible to or may become susceptible to (or at risk of) infection by a pathogenic fungus, such as one or more of the Pneumocystis, Aspergillus, Candida, or Cryptococcus fungal pathogens, either in or outside of a nosocomial environment. By way of example, such a patient may be preparing to undergo a transplant (a pre-transplant patient) or may have received a transplant (a post-transplant patient) and is administered one or more immunosuppressive drugs or medications (anti-rejection medications) and/or is otherwise treated with drugs to reduce the likelihood of rejection of the transplanted organ or tissue, thereby making the patient more vulnerable, susceptible to, or at risk of infection and/or disease caused by one or more of the Pneumocystis, Aspergillus, Candida, or Cryptococcus fungal pathogens. Patients having other types of diseases and conditions, such as, without limitation, cancers, immunodeficiency diseases, (HIV, AIDS, HIV/AIDS), autoimmune diseases, rheumatoid arthritis, or psoriasis, and the like, may also be administered medications having an immune suppressive effect to treat or manage their conditions and thus suffer from, or be at risk of, infection by one or more fungal pathogens. Such patients may thus be immunocompromised. Non-limiting classes of immune suppressive drugs and medications include, for example, corticosteroids, such as prednisone (e.g., DELATSONE, ORASONE); budesonide (ENTOCORT EC), or prednisolone (MLLIPRED) calcineurin inhibitors, such as cyclosporine (NEORAL, SANDIMMUNE, SANGCYA); or tacrolimus (ASTAGRAF XL, ENVARSUS XR, PROGRAF); mTOR inhibitors, such as sirolimus (RAPAMUNE), everolimus (AFINITOR, ZORTRESS); Inosine Monophosphate Dehydrogenase (IMDH) inhibitors, such as azathioprine (AZASAN, IMURAN), leflunomide (ARAVA), mycophenolate (CELLCEPT, MYFORTIC); Biologics and monoclonal antibodies or monoclonal antibody-based antibodies or antigen binding fragments thereof, such as abatacept (ORENCIA); adalimumab (HUMIRA); anakinra (KINERET); certolizumab (CIMZIA); etanercept (ENBREL); golimumab (SIMPONI); infliximab (REMICADE); ixekizumab (TALTZ); natalizumab (TYSABRI); rituximab (RITIXAN); secukinumab (COSENTYX); tocilizumab (ACTEMRA); ustekinumab (STELARA); and vedolizumab (ENTYVIO). In an embodiment, the patient is to receive or has received a transplant of an organ selected from kidney, liver, heart, bone marrow, pancreas, lung, gall bladder, bladder, etc.

Immunogenic compositions comprising a non-naturally occurring PF-KEX1b or PF-KEX2b peptide, a polynucleotide encoding the pan-fungal Kex peptide, or antibodies directed against the non-naturally occurring PF-KEX1b or PF-KEX2b peptide (or antiserum containing such antibodies) can be administered to the patient who is receiving transplant rejection medication, or other immune suppressive medication, in an effective amount to heighten, increase, or augment the immune response against disease or symptoms thereof associated with or caused by infection by one, one or more, two or more, three or more, or all four of the Pneumocystis, Aspergillus, Candida, or Cryptococcus fungal pathogens in the immune suppressed patient. In an embodiment, the patient receiving immune suppressing drugs can be evaluated and monitored during treatment with immune suppressive drugs for the presence of antibodies (and antibody titers) against one or more of the fungal pathogens by employing the methods and kits as described herein.

Optionally, an immunogenic composition comprising a non-naturally occurring PF-KEX1b or PF-KEX2b peptide, a polynucleotide encoding the pan-fungal Kex peptide, or an isolated antiserum generated in response to the PF-KEX1b or PF-KEX2b peptide used as an immunogen as described herein, may be administered in combination with one or more of any other treatment or therapy, e.g., anti-fungal therapies. For example, an immunogenic composition comprising a non-naturally occurring PF-KEX1b or PF-KEX2b peptide, a polynucleotide encoding the pan-fungal Kex peptide, or an isolated antiserum or immune plasma generated in response to the non-naturally occurring PF-KEX1b or PF-KEX2b peptide immunogen, wherein the isolated antiserum or immune plasma contains anti-PF-KEX1b or PF-KEX2b peptide antibodies, may be administered to a subject in combination with other antibodies or antibody cocktails with anti-fungal activity (including, for example, immune plasma), or in combination with one or more drugs to provide protective immunity in the recipient against one or more of a Pneumocystis, Aspergillus, Candida, and/or Cryptococcus fungal organism. By way of non-limiting example, one or more drugs having anti-fungal activity include trimethoprim-sulfamethoxazole, azithromycin-sulfamethoxazole, clarithromycin-sulfamethoxazole, atovaquone, sulfadoxine-pyrimethamine, erythromycin-sulfisoxazole, PS-15, and dapsone-trimethoprim, as well as intravenous pentamidine and clindamycin-primaquine. In an embodiment of any of the foregoing, the immunogenic composition comprising a PF-KEX1b or PF-KEX2b peptide, a polynucleotide encoding the pan-fungal Kex peptide, or an isolated antiserum generated in response to the PF-KEX1b or PF-KEX2b peptide immunogen is provided in a pharmaceutically acceptable composition.

In an embodiment of any of the foregoing aspects, the PF-KEX1b or PF-KEX2b peptide immunogen, an immunogenic composition comprising a non-naturally occurring PF-KEX1b or PF-KEX2b peptide, a polynucleotide encoding the pan-fungal Kex peptide, or antiserum containing antibodies generated in response to immunization with the PF-KEX1b or PF-KEX2b peptide immunogen and isolated from a donor subject, can be administered to a recipient subject, allowing the recipient subject to acquire immune protection, including memory immune protection, against infection or disease caused by one or more of the fungal pathogens.

Methods for administering both single and combination therapies (e.g., concurrently or otherwise) are known to those skilled in the art and are described, for example, in Remington's Pharmaceutical Sciences, 12^{th} edition, Edited by E. W. Martin, Mack Publishing Co. In an embodiment, the antiserum provides a therapeutic, antibody-containing composition that treats infection or disease caused by one or more fungal pathogens as described herein. In another embodiment, the antiserum provides prophylactic, antibody-containing composition that prevents and protects against disease, severe disease, and/or symptoms thereof caused by one or more fungal pathogens as described herein. In an embodiment, the isolated antiserum is in a pharmaceutically acceptable composition.

### Additional Methods

At present, there is a dearth of methods as well as reagents to determine if a patient who is asymptomatic for infection by one or more, two or more, three or more, or each of Pneumocystis, Aspergillus, Candida, or Cryptococcus fungal pathogens is susceptible, vulnerable, or at risk for infection by one or more of these pathogens. It is currently difficult to plate out these fungal organisms as they may be present in very low amounts, or they do not grow under the culture conditions available for assessing their presence in a subject. Consequently, it is difficult for a medical practitioner and the patient to know whether the patient is actually infected with one or more of Pneumocystis, Aspergillus, Candida, or Cryptococcus, or if the patient is likely to become infected, for example, after a medical procedure, surgery, or transplant.

It is also difficult to identify, qualify, or stratify patients who are or may be susceptible to infection by one or more fungal pathogens and to monitor patients for changes in susceptibility over time, e.g., during recovery from surgery or during immunosuppressive therapies, for example, following organ transplantation, or during other chemotherapy treatments, or for reduction or elimination of infection in a patient undergoing treatment for a fungal infection or associated disease over time. The methods described herein provide a viable solution for such medical needs. In addition, methods are provided that allow a patient to be treated with an appropriate or a more directed fungal therapy by stratifying patients based on whether they possess or do not possess antibodies specific for a particular fungal pathogen, thereby deterring disease or infection, or based on whether the patient has or does not have anti-fungal Kexin peptide antibodies that are specific for a given type of pathogenic fungus.

In an embodiment, a method is provided for detecting antibodies that bind to or react against a non-naturally occurring PF-KEX1b or PF-KEX2b peptide in a sample obtained from a subject, in which the method comprises: (a) contacting a biological sample obtained from the subject with a non-naturally occurring PF-KEX1b or PF-KEX2b peptide; and (b) detecting antibodies in the sample that specifically bind to the non-naturally occurring PF-KEX1b or PF-KEX2b peptide, wherein the detection of binding indicates the presence of antibodies against the Kex peptide of the fungal organisms in the subject's sample. In an embodiment, the PF-KEX1b and/or PF-KEX2b peptide is attached to a solid support or substrate. In an embodiment, the binding is detected by performing an immunoassay, e.g., an enzyme linked immunosorbent assay (ELISA) or a chip assay.

In another embodiment, a method of monitoring or detecting antibodies to fungal organisms associated with infection in a subject who has undergone a transplant or who is to undergo a transplant procedure to determine, for example, whether the subject is protected or will be protected from infection by one or more fungal pathogens selected from Pneumocystis, Aspergillus, Candida, or Cryptococcus, in which the method comprises: (a) measuring at a first time point the level of antibodies that bind to a non-naturally occurring PF-KEX1b or PF-KEX2b peptide in a sample obtained from the subject prior to undergoing transplant surgery; (b) measuring the levels of antibodies that bind to the non-naturally occurring PF-KEX1b or PF-KEX2b peptide in a sample obtained from the subject at one or more time points after the subject has undergone transplant surgery; and (c) detecting that the subject sample contains a level of antibodies that specifically bind to the non-naturally occurring PF-KEX1b or PF-KEX2b peptide relative to a predetermined or threshold level or to a control level, wherein a high level of antibodies that bind to the non-naturally occurring PF-KEX1b or PF-KEX2b peptide in the subject's sample indicates that the subject has produced an immune response against the fungal organism, e.g., the kexin peptide of the fungal organism. In an embodiment, antibodies detected in the subject's sample that bind to the non-naturally occurring pan-fungal PF-KEX1b or PF-KEX2b peptide may serve to protect the subject from infection by Pneumocystis, Aspergillus, Candida, or Cryptococcus, according to the methods described herein. In embodiments of the foregoing methods, one of both of the PF-KEX1b peptide and/or the PF-KEX2b peptide may be included.

Repeating the practice of the above-described method over time (at different time intervals or different time periods) allows monitoring levels of the subject's antibodies that bind to the non-naturally occurring PF-KEX1b or PF-KEX2b peptide and can inform the medical practitioner or clinician as to whether continued, new, or different treatment of the subject with an appropriate anti-fungal drug or therapy is needed or warranted, or whether no or less anti-fungal treatment is warranted, based on the measured titers of anti-PF-KEX1b or anti-PF-KEX2b peptide antibodies in the subject's sample.

Other embodiments also provide methods for detecting in a subject's biological sample, e.g., blood, serum, plasma, lymph, bronchoalveolar lavage fluid, the presence of antibodies that bind a non-naturally occurring PF-KEX1b or PF-KEX2b peptide, wherein the levels of antibodies against the non-naturally occurring PF-KEX1b or PF-KEX2b peptide in the biological sample are determined simultaneously. For example, in one embodiment, the method comprises: (a) contacting a biological sample obtained from the subject with a non-naturally occurring PF-KEX1b or PF-KEX2b peptide that selectively binds to a plurality of antibodies in the subject's sample for a period of time sufficient to form bound PF-KEX1b or PF-KEX2b peptide-antibody complexes; (b) detecting binding of the PF-KEX1b or PF-KEX2b peptide to the plurality of antibodies in the subject's sample, thereby determining the levels of antibodies to kexin peptide in the sample; and (c) comparing the levels of the plurality of antibodies in the sample with predetermined threshold values, wherein levels of antibodies that bind to at least one of the PF-KEX1b or PF-KEX2b peptides above or below the predetermined threshold values indicates, for example, that the subject has an antibody titer and has generated an immune response against a Kex peptide of one or more of the fungal organisms. Accordingly, the subject having a measured antibody response to the Kex peptide is protected from disease, serious disease, or symptoms thereof associated with or caused by infection by one or more of the Pneumocystis, Aspergillus, Candida, or Cryptococcus fungal organisms.

In another embodiment, methods for assessing antibodies that bind to the Kex peptide of one or more of the fungal organisms Pneumocystis, Aspergillus, Candida, or Cryptococcus in a subject are provided, in which the methods comprise: (a) contacting a biological sample obtained from the subject with a composition comprising the PF-KEX1b peptide and/or the PF-KEX2b peptide for a period of time sufficient to form antibody-Kex peptide complexes; (b) detecting binding of the Kex peptides to antibodies in the sample, thereby detecting the level or titer of anti-Kex peptide antibodies in the sample; and (c) comparing the level or titer of the anti-Kex peptide antibodies in the biological sample with predetermined threshold values or control values, wherein levels of at least one of the anti-Kex peptide antibodies above or below the predetermined threshold values indicates that the subject has or does not have, respectively, an adequate immune response (antibody response) to prevent infection by the one or more fungal organisms.

### Antibodies

As described herein, antisera comprising antibodies that specifically bind a non-naturally occurring PF-KEX1b or PF-KEX2b peptide and that cross-react with a Kexin peptide of one or more different fungal organisms, such as one or more of Pneumocystis, Aspergillus, Candida, or Cryptococcus, provide therapeutic treatment and/or immune protection against disease and symptoms thereof caused by or associated with infection by one or more of these fungal pathogens are thus are useful in therapeutic methods. In particular embodiments, methods of using isolated antiserum (or immune plasma) comprising antibodies, or antigen binding fragments thereof, that specifically bind the non-naturally occurring PF-KEX1b or PF-KEX2b peptide and that cross-react with a kexin peptide of one or more of Pneumocystis, Aspergillus, Candida, or Cryptococcus are provided for the treatment or prevention of disease and/or the symptoms thereof caused by or associated with infection by these fungal pathogens, such as pulmonary diseases and disorders of various types, pneumonia, COPD, aspergillosis, invasive pulmonary aspergillosis (IPA), etc.

Methods of preparing antibodies are well known to those of ordinary skill in the science of immunology. As used herein, the term “antibody” means not only intact antibody molecules, but also fragments of antibody molecules that retain immunogen (immunogenic antigen)-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of an intact antibody, clear more rapidly from the circulation, and may have less nonspecific tissue binding than an intact antibody (Wahl et al., J. Nucl. Med. 24:316-325 (1983)). The antibodies may comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8 (10): 1057-1062, (1995)), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody, because small molecules of ˜60-100 kDa in size provide faster blood clearance and rapid tissue uptake. See, e.g., Power et al., (Generation of recombinant multimeric antibody fragments for tumor diagnosis and therapy, Methods Mol Biol, 207, 335-50, (2003); and Wu et al., Anti-carcinoembryonic antigen (CEA) diabody for rapid tumor targeting and imaging, Tumor Targeting, 4, 47-58, (1999)).

Various techniques for making and using unconventional antibodies have been described. Bispecific antibodies produced using leucine zippers are described by Kostelny et al. (J. Immunol. 148 (5): 1547-1553, (1992)). Diabody technology is described by Hollinger et al. (Proc. Natl. Acad. Sci. USA 90:6444-6448, (1993)). Another strategy for making bispecific antibody fragments using single-chain Fv (sFv) diners is described by Gruber et al. (J. Immunol. 152:5368, (1994)). Trispecific antibodies are described by Tutt et al. (J. Immunol. 147:60, (1991)). Single chain Fv polypeptide antibodies include a covalently linked V_{H}::V_{L} heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, (1988)). See, also, U.S. Pat. Nos. 5,091,513, 5,132,405 and 4,956,778; and U.S. Patent Publication Nos. 20050196754 and 20050196754.

In various embodiments, an antiserum (isolated antiserum) contains antibodies, or antigen binding fragments thereof, that are monoclonal or polyclonal and specifically bind to a non-naturally occurring PF-KEX1b or PF-KEX2b peptide. Also encompassed are methods of obtaining or isolating antibodies from immune serum (antiserum) or immune plasma and producing hybrid or chimeric antibodies therefrom. In such hybrid or chimeric antibodies one pair of heavy and light chains is obtained from a first antibody, while the other pair of heavy and light chains is obtained from a different second antibody. Such hybrids or chimeric antibodies may also be formed using humanized antibody heavy and light chains. Methods for isolating antibodies and producing hybrid or chimeric antibodies are known and practiced by those having skill in the art.

In general, intact antibodies are said to contain “Fc” and “Fab” regions. The Fc regions are involved in complement activation and are not involved in antigen binding. An antibody from which the Fc region has been enzymatically cleaved, or which has been produced without the Fc region, designated an “F(ab′)₂” fragment, retains both antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies (and immune serum or plasma containing antibodies) can be produced or generated by any of the methods known in the art utilizing soluble polypeptides, peptide, or immunogenic fragments thereof, (e.g., PF-KEX1b or PF-KEX2b peptide) as an immunogen. One method of obtaining antibodies is to immunize suitable host animals or subjects with a PF-KEX1b or PF-KEX2b peptide immunogen, or a polynucleotide encoding the pan-fungal Kex peptide, and to follow standard procedures for polyclonal or monoclonal antibody production. In brief, the administered immunogen will facilitate presentation of the immunogen (or immunogenic fragments of the immunogen) on the cell surface. Immunization of a suitable host can be carried out in a number of ways. By way of example, nucleic acid sequences encoding immunogenic a PF-KEX1b or PF-KEX2b peptide can be provided to the host in a delivery vehicle (or a molecular expression construct) that is taken up by immune cells of the host. The cells will, in turn, process and appropriately express the PF-KEX1b or PF-KEX2b peptide in a manner that generates an immunogenic response in the host. In embodiments, non-naturally occurring PF-KEX1b or PF-KEX2b peptide may be expressed by the delivery vehicle or expression construct. In other exemplary embodiments, nucleic acid sequences encoding a non-naturally occurring PF-KEX1b or PF-KEX2b peptide may be expressed in cells in vitro, and the expressed, recombinant PF-KEX1b or PF-KEX2b peptide products may be isolated and used as immunogens to raise anti-Kex peptide antibodies and to generate an anti-Kex peptide antiserum in a suitable immunized host.

Alternatively, antibodies against non-naturally occurring PF-KEX1b or PF-KEX2b peptide may, if desired, be derived from an antibody phage display library. A bacteriophage is capable of infecting and reproducing within bacteria, which can be engineered, when combined with human immunoglobulin (antibody) genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibody purification methods include, without limitation, salt precipitation (for example, with ammonium sulfate), ion exchange chromatography (for example, on a cationic or anionic exchange column, preferably run at neutral pH and eluted with step gradients of increasing ionic strength), gel filtration chromatography (including gel filtration HPLC) and chromatography on affinity resins such as protein A, protein G, hydroxyapatite, or anti-immunoglobulin.

In certain aspects, antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can, in turn, be obtained from the hybridoma that produces the antibody, and then the antibody may be produced synthetically or recombinantly from these nucleic acid sequences. To produce large amounts of antibody, it is generally more convenient to obtain an ascites fluid. The method of raising ascites fluid generally comprises injecting hybridoma cells into an immunologically naïve histocompatible or immunotolerant mammal, especially a mouse. The mammal may be primed for ascites production by prior administration of a suitable composition (e.g., Pristane). Ascites fluid containing antibodies, typically in high concentration, can be obtained from the peritoneal fluid of the animal that harbors the injected hybridoma cells.

Monoclonal antibodies (Mabs) can also be “humanized” by methods known in the art. “Humanized” antibodies are antibodies in which at least part of the sequence has been altered from its initial form to render it more like immunoglobulins derived from a human source. Techniques to humanize antibodies are particularly useful when antibodies are generated in a non-human animal (e.g., mice, rats). Nonlimiting examples of methods for humanizing a murine antibody are provided, for example, in U.S. Pat. Nos. 4,816,567, 5,530,101, 5,225,539, 5,585,089, 5,693,762 and 5,859,205.

In an embodiment of the foregoing, one or more antibodies or antigen binding fragments thereof generated against the non-naturally occurring PF-KEX1b or PF-KEX2b peptide can be used in a pharmaceutical composition alone or in combination to provide immune treatment and/or protection against disease or symptoms thereof caused by one or more of the described fungal pathogens in a subject in need thereof. Such antibodies may be isolated or purified from an antiserum as described herein, or they may be generated, e.g., by recombinant molecular biology techniques, purified and formulated for pharmaceutical use in a subject in need. Such a formulation of antibodies that bind to the PF-KEX1b or PF-KEX2b peptide may have immune protective properties similar to those afforded by an isolated antiserum comprising anti-fungal Kex peptide antibodies.

### Vaccines

A vaccine is a biological preparation that provides active, acquired immunity (e.g., protective immunity) to a particular disease in a subject. A vaccine typically contains an agent that resembles a disease-causing pathogenic agent, e.g., a microorganism, a fungus, etc., and is often made from a weakened or killed form of the agent, or a toxin or surface protein, peptide, or encoding polynucleotide thereof, of the agent. After administration of the vaccine to a subject, the agent is expressed and recognized as foreign (or “non-self”) to the subject and stimulates the subject's immune system to mount an immune response (a B cell (antibody) and/or a T cell (cellular) immune response) and to destroy the agent. In addition, cells (e.g., B cells) of the immune system that are exposed to the vaccinating agent retain a memory of the agent, such that the agent is recognized and destroyed by antibodies produced by the memory cells upon a later or subsequent encounter. Vaccines can be prophylactic (e.g., to prevent or ameliorate the effects of a future infection by a pathogen), or therapeutic (e.g., to treat disease or infections caused by or associated with pathogens or disease-causing agents upon or after a subject has been infected with or encountered a pathogen).

While many vaccines are prepared from an attenuated version of a pathogen or from inactivated disease-causing organisms, or a suitable part of such pathogens or organisms, such as a toxin, protein/peptide (e.g., a non-naturally occurring PF-KEX1b or PF-KEX2b peptide), or deleterious enzyme, the antigen to which the immune system responds frequently constitutes a relatively small number of amino acids, such as a peptide that retains immunogenicity and antigenicity. A protein or peptide part of a pathogen may constitute a vaccine. A peptide vaccine is any peptide which serves to immunize an organism (elicit an immune response or a protective immune response, such as an antibody (B cell) response and/or an immune cell (T cell) response in the immunized organism) against a pathogenic agent, protein, peptide, toxin, or pathogen. In embodiments, the peptide antigen may be the non-naturally occurring PF-KEX1b or PF-KEX2b peptide. In an embodiment, a vaccine comprising a non-naturally occurring PF-KEX1b or PF-KEX2b peptide antigen may be used to provide immune protection against the Pneumocystis, Aspergillus, Candida, or Cryptococcus fungal organisms following administration to a recipient subject in need.

For non-attenuated vaccines, the peptide sequences that trigger a protective immune response are identified, and synthetic (or recombinantly-produced) versions of the peptides are employed as the vaccine substance. Because they are non-naturally occurring and synthetic, peptide vaccines pose little to no risk of mutation or reversion, and little or no risk of contamination by pathogenic or toxic substances. Moreover, chemical manipulation or modification of the peptide structure may result in increased stability and decreased unwanted side effects or adverse effects that may be associated with a native protein or peptide sequence.

Synthetically or recombinantly produced peptide antigens can be readily prepared in large amounts as components of vaccines. Such substances may also expose parts of a protein antigen that are not recognized by the immune system during a natural infection, possibly as a result of masking or post-translational modifications of proteins. Sequencing new strains and serotypes of microorganisms, fungal pathogens and other pathogenic organisms allows for rapid modification of peptide antigens to generate strain-specific immune responses, particularly against an antigenic epitope that is recognized and targeted by antibodies and cells of the host's or recipient's immune system. In some cases, modelling of three-dimensional epitopic or antigenic sites of a pathogen may be employed to generate synthetically the correct epitopic or antigenic site(s) on peptide antigens.

In an aspect, a vaccine (or an immunogenic composition) is provided, which comprises a synthetically (recombinantly) produced peptide, i.e., a PF-KEX1b or PF-KEX2b peptide, that is nonidentical, but immunologically targetable, among several different types of fungal pathogens (e.g., the Pneumocystis, Aspergillus, Candida and Cryptococcus fungal pathogens) and is useful for treating or preventing disease caused by or associated with one or more than one fungal pathogen after administration (immunization) to a subject. In an embodiment, a peptide vaccine or immunogenic composition comprising a non-naturally occurring PF-KEX1b or PF-KEX2b peptide, when used to immunize an individual, elicits an immune response in the form of the production of antiserum (or immune plasma) containing antibodies which cross-react with and cross-protect against the kex peptides of etiologically distinct fungal pathogens Pneumocystis, Aspergillus, Candida and Cryptococcus, and in particular, Pneumocystis hominis, Aspergillus fumigatus, Candida albicans and Cryptococcus neoformans. Accordingly, an antiserum or immune plasma generated by a vaccine or immunogenic composition comprising a non-naturally occurring PF-KEX1b or PF-KEX2b peptide may be used as a sole therapeutic or protective agent needed to treat or prevent disease or symptoms thereof caused by or associated with more than one kexin-producing fungal pathogen, namely, the Pneumocystis, Aspergillus, Candida and/or Cryptococcus fungal pathogens, and in particular, infection or disease caused by or associated with Pneumocystis hominis, Aspergillus fumigatus, Candida albicans and/or Cryptococcus neoformans. In an embodiment, the antiserum generated by such a peptide vaccine is isolated. In an embodiment, the isolated antiserum is used in a pharmaceutical composition.

In some aspects, a genetic vaccine is provided. A genetic vaccine is any vaccine that comprises a polynucleotide sequence encoding an immunogen, wherein the immunogen, once expressed, serves to immunize an organism. Administration of a genetic vaccine elicits an immune response or a protective immune response, such as an antibody (B cell) response and/or an immune cell (T cell) response in the immunized organism against a pathogen or product produced by a pathogen after the immunogen is expressed in a cell. In some embodiments, the genetic vaccine provides a polynucleotide sequence encoding a Kex peptide, such as a PF-KEX1b or PF-KEX2b peptide described herein. In some embodiments, the genetic vaccine provides a polynucleotide sequence encoding the PF-KEX1b peptide and the PF-KEX2b peptide. In some embodiments, the polynucleotide encoding the PF-KEX1b or PF-KEX2b peptide resides in a vector having elements, such as promoters and enhancers, to facilitate expression of the encoded PF-KEX1b or PF-KEX2b peptide. In some cases, the nucleic acid in a genetic vaccine may be integrated into the subject's genome, wherein expression of the immunogen may be driven by an endogenous promoter or a promoter encoded by the inserted nucleic acid. In other cases, the nucleic acid in a genetic vaccine is not integrated into the subject's genome. The polynucleotide encoding the immunogen in a genetic vaccine may be a DNA polynucleotide or a RNA polynucleotide. The polynucleotide may include a nucleotide analog, which can inhibit degradation of the polynucleotide.

### Pharmaceutical Compositions

Also featured herein are methods of treating or preventing disease and/or the symptoms thereof associated with or caused by infection of a subject by one or more fungal pathogens. In an aspect, the methods include administering to a subject in need thereof an immunologically effective amount of a PF-KEX1b or PF-KEX2b peptide, an immunogenic composition comprising a PF-KEX1b or PF-KEX2b peptide, a polynucleotide encoding the PF-KEX1b or PF-KEX2b peptide, or an isolated antiserum generated in response to immunization with the non-naturally occurring PF-KEX1b or PF-KEX2b peptide, which treats and/or protects the subject from disease and/or the symptoms thereof associated with infection by at least one of the different fungal pathogens selected from Pneumocystis, Aspergillus, Candida, or Cryptococcus. In an embodiment, the isolated antiserum is used in a pharmaceutical composition.

Typically, the carrier or excipient for an immunogenic composition or vaccine as described herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intravenous, intranasal, and the like. Such methods also include administering an adjuvant, such as an oil-in-water emulsion, a saponin, a cholesterol, a phospholipid, a CpG, a polysaccharide, variants thereof, and a combination thereof, with a composition as described herein. Optionally, a formulation for prophylactic administration also contains one or more adjuvants for enhancing the immune response to an antigen or immunogen, such as a PF-KEX1b or PF-KEX2b peptide antigen or immunogen. Suitable adjuvants include, without limitation, complete Freund's adjuvant, incomplete Freund's adjuvant, saponin, alpha-galactosylceramide (α-GC), alum, ALHYDROGEL®, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59. In an embodiment, the isolated antiserum is used in a pharmaceutical composition.

The administration of an immunogenic composition comprising a PF-KEX1b or PF-KEX2b peptide, a polynucleotide encoding the pan-fungal Kex peptide, or an antiserum, such as an isolated antiserum, monoclonal or polyclonal antibodies against the PF-KEX1b or PF-KEX2b peptide as a therapeutic for the treatment or prevention of disease, severe or serious disease, or symptoms thereof caused by or associated with an infection by a fungal pathogen as described herein (e.g., pulmonary infection or disease, poor pulmonary function, COPD, pneumonia, aspergillosis, Invasive Pulmonary Aspergillosis (IPA), etc.) may be by any suitable means that results in a concentration of the therapeutic that, combined with other components, if desired, is effective in ameliorating, reducing, eliminating, abating, diminishing, or stabilizing disease or disease symptoms in a subject. The therapeutic may be administered systemically, for example, formulated in a pharmaceutically-acceptable composition or buffer such as physiological saline. Preferable routes of administration include, for example, subcutaneous, intravenous, intraperitoneal, intramuscular, intrathecal, or intradermal injections, e.g., that provide continuous, sustained levels of the therapeutic in the subject. The amount of the therapeutic to be administered varies depending upon the manner of administration, the age and body weight of the subject, and with the disease and/or clinical symptoms associated with the fungal infection. Generally, amounts will be in the range of those used for other agents employed in the treatment of pulmonary disease or dysfunction, although in certain instances, lower amounts may be suitable because of the increased range of protection and treatment afforded by the therapeutic. A composition is administered at a dosage that ameliorates, decreases, diminishes, abates, alleviates, or eliminates the effects of the fungal pathogen infection or disease (e.g., pulmonary infection and disease and the symptoms thereof) as determined by a method known to one skilled in the art. In an embodiment, an isolated antiserum is administered or provided to a recipient subject at or near a site of the infection or colonization by the pathogenic fungal organism or organisms.

In embodiments, a therapeutic or prophylactic treatment agent may be contained in any appropriate amount in any suitable carrier substance, and is generally present in an amount of 0.1%-95%, 0.5%-95%, or 1%-95% by weight of the total weight of the composition. The composition may be provided in a dosage form that is suitable for a parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent or drug to a particular cell type or tissue. For some applications, controlled release formulations obviate the need for frequent dosing during the day to sustain a therapeutic level in plasma, serum, or blood. In an embodiment, an isolated antiserum may be formulated with one or more additional components for administration to a subject.

Any of a number of strategies can be pursued to obtain controlled release in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, noted supra.

Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease or dysfunction, such as pulmonary disease or dysfunction, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic (e.g., an immunogenic composition comprising a non-naturally occurring PF-KEX1b peptide or PF-KEX2b peptide, a polynucleotide encoding the pan-fungal Kex peptide, or an isolated anti-fungal antiserum generated in response to immunization with the non-naturally occurring PF-KEX1b peptide or PF-KEX2b peptide as described herein) is formulated for intravenous delivery, e.g., intravenous, injection, or intrathecal delivery. In an embodiment, the antiserum is an isolated antiserum. To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. The aqueous formulation may also contain one or more preservatives (e.g., methyl, ethyl or n-propyl p-hydroxybenzoate). In cases in which one of the agents is only sparingly or slightly soluble in water, a dissolution enhancing or solubilizing agent can be added, or the solvent may include 10-60% w/w of propylene glycol or the like.

Kits and Compositions for Detecting and/or Quantifying Antibodies that React with PF-KEX1b Peptide or PF-KEX2b Peptide

In another embodiment, kits and compositions are provided that advantageously allow for the detection and/or quantification of the presence of antibodies directed against the Kex protein or peptide of one or more of Pneumocystis, Aspergillus, Candida, or Cryptococcus fungal pathogens, or the levels of such one or more antibodies that may be present, in a subject's sample (e.g., blood or serum). In an embodiment, the subject is a human patient. In an embodiment, the patient has undergone a transplant, e.g., an organ or tissue transplant, or is to undergo a transplant, and thus may be at higher risk for infection by one or more fungal pathogens. In an embodiment, the transplant patient, or the patient to undergo a transplant, is immunosuppressed and/or is otherwise treated with drugs to reduce the likelihood of rejection of the transplanted organ or tissue, thereby making the patient more vulnerable or susceptible to infection and/or disease caused by one or more of the Pneumocystis, Aspergillus, Candida, or Cryptococcus fungal pathogens. In an embodiment, the patient has received, or is to receive, a transplant of an organ selected from kidney, liver, heart, bone marrow, pancreas, lung, etc.

Such kits as described herein fulfill a long-felt need in the art for detecting or qualifying whether any patient, but particularly a transplant patient, has adequate levels (titer) of anti-fungal pathogen antibodies to ensure that the patient does not succumb to disease and/or is treated and/or protected following infection by one or more fungal pathogens as described herein, for example, during a hospital stay, or during or following a medical procedure or treatment (e.g., surgery or transplant), performed either on in-patient or an out-patient basis. At present, because of a lack of appropriate reagents and assays, it is difficult to assess whether a patient who is to undergo a medical procedure or surgery, in particular, an immunosuppressed patient who is to undergo a transplant procedure, or a patient who is to initiate other immunosuppressive therapies, will contract a fungal infection, e.g., infection and/or disease caused by one or more of the Pneumocystis, Aspergillus, Candida, or Cryptococcus fungal pathogens following or during immunosuppressive therapies and treatments. The use of a kit with which a patient's sample can be tested to determine if the patient has an antibody titer against one or more of these fungal pathogens (e.g., a high or a low antibody titer against the kexin peptide of one or more of the fungal pathogens) would greatly enhance the success of the patient's post-surgical or post-transplant recovery and directed treatment. For example, if, following testing of a patient's sample (e.g., a blood, plasma, or serum sample from a transplant patient) using a kit as described herein, the patient is determined to have a low, negligible, or no antibody (antiserum) titer against one or more of the fungal pathogens, in particular, against the Kex peptide of one or more of the fungal pathogens, it could be surmised that the patient would not be naturally or adequately protected against a possible or real infection by one or more of the Pneumocystis, Aspergillus, Candida, or Cryptococcus fungal pathogens,

A kit as described herein would allow the tester and the patient to determine and know if the patient's sample (serum or plasma sample) contains antibodies against one or more, two or more, three or more, or four of Kex protein/peptide of the Pneumocystis, Aspergillus, Candida, or Cryptococcus organisms. Should the results obtained from the use of the kit indicate that the patient has no specific anti-fungal Kex peptide antibodies, or a low titer of such antibodies (e.g., no specific anti-fungal Kex peptide antibodies in serum or plasma), directed to a specific anti-fungal Kex peptide, the patient would be identified as potentially vulnerable or susceptible to disease or serious disease following infection by a particular fungal pathogen and could then be administered the appropriate anti-fungal treatment for the specific fungal pathogen against which the patient has no, or negligible, specific antibodies, or a reduced antibody titer. In an embodiment, the patient is administered a prophylactic anti-fungal treatment or therapy. In an embodiment, the treatment comprises administering to the patient an appropriate drug or medication that is best designed to treat infection or disease associated with infection by a specific fungal pathogen or by two or more fungal pathogens, namely, Pneumocystis, Aspergillus, Candida, or Cryptococcus. In an embodiment, the treatment comprises administering to the patient a composition as described herein comprising a non-naturally occurring PF-KEX1b peptide or PF-KEX2b peptide or a polynucleotide encoding the pan-fungal Kex peptide to generate a cross-reactive (cross-protective) antibody immune response in the patient, thereby reducing or eliminating disease, serious disease, and/or the symptoms thereof caused by or associated with infection by one or more of the Pneumocystis, Aspergillus, Candida, or Cryptococcus organisms. Antibodies produced against a non-naturally occurring PF-KEX1b peptide or PF-KEX2b peptide described herein can recognize a Kex peptide of the other fungal organisms as described herein, thereby conferring treatment and/or protection (cross-protection) against more than one of the fungal organisms in the patient.

In an embodiment, a kit is provided for detecting, or qualifying the levels of, antibodies directed against fungal Kex protein or peptides of Pneumocystis, Aspergillus, Candida, or Cryptococcus organisms in a patient's biological sample, in which the kit comprises a substrate having attached thereto a non-naturally occurring PF-KEX1b peptide and/or PF-KEX2b peptide for measuring the levels of antibodies in the sample. The substrate is contacted with the biological sample obtained from a patient, and a labeled detection molecule is used to detect and measure the level of antibodies that bind to the PF-KEX1b peptide and/or PF-KEX2b peptide on the substrate. In an embodiment, detecting anti-fungal Kex peptide antibodies in the sample or the measuring the level of such antibodies present in the patient's sample is compared to a positive and/or a negative control. In an embodiment, detecting anti-fungal Kex peptide antibodies in the sample or the measuring the level of such antibodies present in the patient's sample is compared to a cutoff value. In an embodiment, the substrate has attached thereto a non-naturally occurring PF-KEX1b peptide and/or PF-KEX2b peptide and a Kex peptide derived from each of Pneumocystis, Aspergillus, Candida, and Cryptococcus. In an embodiment, the substrate has attached thereto a non-naturally occurring PF-KEX1b peptide and/or PF-KEX2b peptide and Kex peptides derived from one of Pneumocystis, Aspergillus, Candida, and Cryptococcus. In an embodiment, the substrate has attached thereto the PF-KEX1b peptide and/or PF-KEX2b peptide and Kex peptides derived from Pneumocystis and Aspergillus. In an embodiment, the substrate has attached thereto the PF-KEX1b peptide and/or PF-KEX2b peptide and Kex peptides derived from Pneumocystis and Candida. In an embodiment, the substrate has attached thereto the PF-KEX1b peptide and/or PF-KEX2b peptide and Kex peptides derived from Pneumocystis and Cryptococcus. In an embodiment, the substrate has attached thereto the PF-KEX1b peptide and/or PF-KEX2b peptide and Kex peptides derived from Candida and Aspergillus. In an embodiment, the substrate has attached thereto the PF-KEX1b peptide and/or PF-KEX2b peptide and Kex peptides derived from Cryptococcus and Aspergillus. In an embodiment, the substrate has attached thereto the PF-KEX1b peptide and/or PF-KEX2b peptide and Kex peptides derived from Candida and Cryptococcus. In an embodiment, the substrate has attached thereto the PF-KEX1b peptide and/or PF-KEX2b peptide and Kex peptides derived from Pneumocystis, Aspergillus and Candida. In an embodiment, the substrate has attached thereto the PF-KEX1b peptide and/or PF-KEX2b peptide and Kex peptides derived from Pneumocystis, Aspergillus and Candida. In an embodiment, the substrate has attached thereto the PF-KEX1b peptide and/or PF-KEX2b peptide and Kex peptides derived from Pneumocystis, Aspergillus and Cryptococcus. In an embodiment, the substrate has attached thereto the PF-KEX1b peptide and/or PF-KEX2b peptide and Kex peptides derived from Pneumocystis, Cryptococcus and Candida. In an embodiment, the substrate has attached thereto the PF-KEX1b peptide and/or PF-KEX2b peptide and Kex peptides derived from Aspergillus, Cryptococcus and Candida. In an embodiment, the PF-KEX1b peptide and/or PF-KEX2b peptide are recombinantly produced. In an embodiment, the detection of antibodies in the sample that bind to the fungal-derived Kex peptide is performed using an immunoassay, such as an ELISA. In an embodiment, the ELISA detects a complex between a Kex peptide bound to an anti-fungal Kex peptide antibody present in the sample. In an embodiment, the detection of antibodies in the sample that bind to the fungal-derived Kex peptide is performed using an immunosorbent assay, by immunoprecipitation, by immunoblotting, or a combination thereof.

Also provided are kits comprising reagents that allow for assessing, measuring, evaluating or detecting antibodies directed against the PF-KEX1b peptide and/or PF-KEX2b peptide. Such antibodies may be contained in a biological sample obtained from a subject undergoing testing, assessment, or evaluation using the kit. In particular, the biological sample may be a blood, serum, plasma, urine, cerebrospinal fluid, sputum, bronchiolar lavage, tears, saliva, stool, or semen sample, or tissue or cell sample obtained from a subject. In particular, the reagents of the kit comprise the PF-KEX1b peptide and/or PF-KEX2b peptide.

In a specific embodiment, the kit is provided as an enzyme linked immunosorbent assay (ELISA) kit comprising the PF-KEX1b peptide and/or PF-KEX2b peptide. In other embodiments, the provided kit allows for the detection of cross-reactive antibodies, wherein the antibodies are produced by immunization with the PF-KEX1b peptide and/or PF-KEX2b peptide. In such embodiments, the kit is provided as an ELISA kit comprising the Kex peptides of Pneumocystis, Aspergillus, Candida, or Cryptococcus attached to a solid support or substrate. The peptides attached to the substrate thus perform as “capture” reagents that bind to antibodies present in the sample obtained from a subject undergoing testing. By way of example, the ELISA kit may comprise a solid support, such as a chip, microtiter plate comprising many wells (e.g., a 96-well plate), bead, or resin having the peptide capture reagents attached thereon. In one embodiment, the kit comprises a Kex peptide derived from each of Pneumocystis, Aspergillus, Candida, or Cryptococcus as described herein attached independently to discrete areas or components of solid substrates or supports, for example, the Kex peptides of each fungal organism are attached to separate and discrete wells of a microtiter plate or are independently attached to beads to produce populations of beads having the Kex peptides from each of Pneumocystis, Aspergillus, Candida, or Cryptococcus attached. In another embodiment, the kit comprises a combination or mixture of the Kex peptides derived from Pneumocystis, Aspergillus, Candida, or Cryptococcus attached to an area or component of the solid substrate or support, for example, the Kex peptides of all of Pneumocystis, Aspergillus, Candida, or Cryptococcus are attached to a single well of a microtiter plate or to a single bead. In a further embodiment, the kit comprises a combination of one, two or more, three or more, or four of the Kex peptides derived from Pneumocystis, Aspergillus, Candida, or Cryptococcus attached to a given area of a solid substrate or support, such as a single well of a microtiter plate.

In embodiments, in the ELISA platform, a well of a microtiter plate may have attached thereto a PF-KEX1b peptide and/or a PF-KEX2b peptide, an Aspergillus Kex peptide, a Candida Kex peptide, or a Cryptococcus Kex peptide. In the ELISA platform, a well of a microtiter plate may have attached thereto a Pneumocystis Kex peptide and an Aspergillus Kex peptide. In the ELISA platform, a well of a microtiter plate may have attached thereto a Pneumocystis Kex peptide and a Candida Kex peptide. In the ELISA platform, a well of a microtiter plate may have attached thereto a Pneumocystis Kex peptide and a Cryptococcus Kex peptide. In the ELISA platform, a well of a microtiter plate may have attached thereto an Aspergillus Kex peptide and a Candida Kex peptide. In the ELISA platform, a well of a microtiter plate may have attached thereto an Aspergillus Kex peptide and a Cryptococcus Kex peptide. In the ELISA platform, a well of a microtiter plate may have attached thereto a Candida Kex peptide and a Cryptococcus Kex peptide. In the ELISA platform, an individual well of a microtiter plate may have attached thereto a Pneumocystis Kex peptide, an Aspergillus Kex peptide and a Candida Kex peptide. In the ELISA platform, an individual well of a microtiter plate may have attached thereto a Pneumocystis Kex peptide, an Aspergillus Kex peptide and a Cryptococcus Kex peptide. In the ELISA platform, an individual well of a microtiter plate may have attached thereto a Pneumocystis Kex peptide, a Candida Kex peptide and a Cryptococcus Kex peptide. In the ELISA platform, an individual well of a microtiter plate may have attached thereto an Aspergillus Kex peptide, a Candida Kex peptide and a Cryptococcus Kex peptide.

The kit may further comprise a means for detecting the peptides or any antibodies bound thereto, e.g., detectable antibodies, a secondary antibody-signal complex, such as horseradish peroxidase (HRP)-conjugated goat anti-rabbit IgG antibody or tetramethyl benzidine (TMB) as a substrate for HRP.

In another embodiment, the kit may be provided as an immunochromatography strip comprising a membrane on which the one, two, three, or four fungal Kex peptides are immobilized, either at discrete loci on the membrane or in combination at one locus of the membrane, and a means for detecting the binding of antibody in a test sample, e.g., detectably labeled peptides, or gold particle bound secondary antibodies, in which the membrane may be a nitrocellulose-based (NC) membrane, a PVDF membrane, or other suitable type of membrane used in the art. The kit may comprise a plastic plate or substrate onto which a sample is applied and immobilized detection agents, such as detectably labeled Kex peptides, e.g., gold particle-bound peptides temporally spaced and immobilized on the substrate, e.g., a glass fiber filter or a nitrocellulose membrane, or a labeled detection agent that can detect a complex of antibody bound to Kex peptide in one or more bands on the substrate. In such a platform, a continuous capillary flow of sample, e.g., blood or serum, is maintained over the detection reagents immobilized on the substrate such that sample antibody bound to labeled Kex peptide or sample antibody complexed to Kex peptide reagent may be detected. In general, ELISA assays and immunosorbent assays, including ELISA membrane-based immunosorbent assays, as well as variations of these assays, are known and practiced by those having skill in the art.

Solid or solid phase substrates, or carriers, that can be effectively used in such assays are well known to those of skill in the art and include, for example, 96-well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Patent Application Publication No. US 2010/0093557 A1. Methods for the automation of immunoassays are well known in the art and include, for example, those described in U.S. Pat. Nos. 5,885,530, 4,981,785, 6,159,750 and 5,358,691.

In an embodiment, a multiplex assay, such as a multiplex ELISA, can be used to detect simultaneously different specific antibodies in a test sample. In embodiments, such methods employ an array, wherein multiple binding agents (for example, capture peptides) specific for multiple antibodies are immobilized on a substrate, such as a membrane, with each capture agent being positioned at a specific, pre-determined, location on the substrate. Methods for performing assays employing such arrays include those described, for example, in U.S. Patent Application Publication Nos. US 2010/0093557A1 and US 2010/0190656A1, the disclosures of which are specifically incorporated by reference herein. If flow cytometry, chemiluminescence, or electron-chemiluminescence technology is employed, multiplex arrays can be used in several different formats. Illustratively, flow cytometric multiplex arrays, also known as bead-based multiplex arrays, include the Cytometric Bead Array (CBA) system from BD Biosciences (Bedford, MA) and multi-analyte profiling (xMAP®) technology from Luminex Corp. (Austin, TX), both of which employ bead sets which are distinguishable by flow cytometry, as well as others known and used in the art.

In another embodiment, a multiplex ELISA from Quansys Biosciences (Logan, UT) involves coating multiple specific capture reagents at multiple spots (one reagent at one spot) in the same well on a 96-well microtiter plate. Chemiluminescence technology is then used to detect multiple antibodies that bind at the corresponding spots on the plate.

In certain embodiments, a patient can be diagnosed by adding a biological sample (e.g., blood, plasma, or serum) from a patient to the kit, or components thereof, and detecting the relevant sample antibodies that specifically bind to the Kex peptide reagents. By way of example, the method comprises: (i) collecting a blood, plasma, or serum sample from the subject; (ii) adding subject's sample to the components in the kit, e.g., a holding tube or a substrate; and (iii) detecting the peptide reagents to which the sample antibodies have bound. In this method, the subject's sample, e.g., blood, plasma, or serum, is brought into contact with the Kex peptide reagent(s), e.g., the PF-KEX1b peptide and/or PF-KEX2b peptide. If the anti-Kex peptide antibody (ies) are present in the sample, the antibodies will bind to the Kex peptide reagents, or a subset thereof. In other kit and diagnostic embodiments, blood is not collected from the patient (i.e., it is already collected), and is assayed for the presence of antibodies that bind to/react with the PF-KEX1b peptide and/or the PF-KEX2b peptide. In some embodiments, sample antibodies generated against one or more, two or more, three or more, or four of the kexin peptides or proteins of Pneumocystis, Aspergillus, Candida, or Cryptococcus organisms bind to or react with the PF-KEX1b peptide or PF-KEX2b peptide using the kit. Moreover, in other embodiments, the sample may comprise a tissue sample or a clinical sample, which can be processed, e.g., homogenized and/or suspended in medium or buffer, prior to assay. In embodiments, any antibody (ies) found to be present in a test sample from a subject may be isolated, or isolated and purified, and further characterized.

The kit can also comprise a washing solution or instructions for making a washing solution, in which the combination of the capture reagents and the washing solution allows capture of anti-Kex antibodies on the solid support for subsequent detection by, e.g., secondary antibodies, labeled reagent peptides, or mass spectrometry. In a further embodiment, a kit can comprise instructions for suitable operational parameters in the form of a label or separate insert (package insert). For example, the instructions may inform a consumer or user about how to collect the sample, how to wash the anti-Kex peptide antibody and Kex peptide reagent complex after binding has occurred, how to interpret the results, etc. In yet another embodiment, the kit can comprise one or more containers with appropriate positive and negative controls or control samples, to be used as standard(s) for detection, calibration, or normalization.

In another aspect, kits are provided for the treatment or prevention of an infection or disease caused by or associated with two or more of Pneumocystis, Aspergillus, Candida, or Cryptococcus fungal pathogens. In some embodiments, the kit includes an effective amount of a therapeutic or prophylactic antiserum, which contains anti-Kex peptide antibodies or antigen binding fragments thereof that bind/react with the PF-KEX1b peptide and/or the PF-KEX2b peptide. In some embodiments, these antibodies cross react with one or more of the kexin peptides or polypeptides of Pneumocystis, Aspergillus, Candida, or Cryptococcus, in unit dosage form. In an embodiment, the antiserum is an isolated antiserum. In other embodiments, the kit includes a therapeutic or prophylactic composition containing an effective amount of an anti-fungal immunoprotective agent such as antiserum in unit dosage form. In some embodiments, the kit comprises a device (e.g., nebulizer, metered-dose inhaler) for dispersal of the composition or a sterile container which contains a pharmaceutical composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition is provided together with instructions for administering the pharmaceutical composition containing isolated antiserum to a subject having or at risk of contracting or developing a disease and/or the symptoms thereof caused by infection by one or more of Pneumocystis, Aspergillus, Candida, or Cryptococcus fungal organisms. The instructions will generally include information about the use of the composition for the treatment or prevention of a disease and/or the symptoms thereof caused by infection by one or more of Pneumocystis, Aspergillus, Candida, or Cryptococcus fungal organisms. In other embodiments, the instructions include at least one of the following: description of the therapeutic/prophylactic agent; dosage schedule and administration for treatment or prevention of disease or symptoms thereof caused by one or more of Pneumocystis, Aspergillus, Candida, or Cryptococcus fungal organisms; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the presently described embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides such as those described herein, and, as such, may be considered in making and practicing the aspects and embodiments described herein. Particularly useful techniques for particular embodiments will be discussed in the sections that follow.

### EXAMPLES

The following examples are put forth to provide those of ordinary skill in the art with a complete disclosure and description of how to make and use the products, compositions, assay, screening, and therapeutic methods as described herein, and are not intended to limit the scope of the described aspects and embodiments.

### Example 1: Pan-Fungal Immunogenic Peptides

Described herein are pan fungal Kexin peptides for use as immunogens in generating a potent immune response against more than one fungal pathogen. The peptides are immunogenic (elicit an anti-peptide antibody response) when administered to a subject in vivo. The peptides include the following:

Pan-fungal peptide 1b, (PF-KEX1b) having the following amino acid sequence:
DDDGKTVDGPSPLVLRAFINGVNNGRNGLGSIYVFASGNGGIYEDNSNFD
GYANSVFTITIGGIDKHGKRPKYSEASSSQLAVTYAGGSA (SEQ ID NO: 1), or an immunogenic fragment thereof.

Pan-fungal peptide 2b, (PF-KEX2b) having the following amino acid sequence:
PDDGKTMEGPDILVLRAFINGVQNGRDGKGSIYVFASGNGGGFEDNSNFD
GYTNSIYSITVGAIDRKGLHPSYSEASSAQLVVTYSSGSG (SEQ ID NO: 2), or an immunogenic fragment thereof.

In an embodiment, the PF-KEX1b and PF-KEX2b peptides comprising serine residues at positions 47 and 77 may provide beneficial properties, e.g., stability, lack of or reduced cross-linking and/or aggregation, for manufacturing and formulating the peptides, for example, for commercial production and use.

The PF-KEX1b and PF-KEX2b peptide sequences are related, but are not identical to, Pan-fungal consensus sequences of Kex1 identified by multisequence alignments of KEX1 peptide sequences from Pneumocystis (Accession No. EU918304.1) (isolated from macaque) Aspergillus fumigatus Kexin (Accession no. XM746441), Candida albicans Kexin (Accession no. AF022372), and Cryptococcus neoformans Kexin (Accession no. XP572303.1) performed using Clustal Omega (http://www.ebi.ac.uk/Tools/msa/clustalo/) to analyze sequence identity and similarity, as described in WO 2020/154510, published Jul. 30, 2020, the entire contents of which are incorporated by reference herein. The 90-mer pan-fungal consensus KEX1 peptides (Pan-fungal peptide 1 and Pan-fungal peptide 2) in WO 2020/154510 had approximately 97% and 69% amino acid sequence identities, respectively, with the amino acid sequence of Pneumocystis KEX1 isolated from humans. The PF-KEX1b peptide (SEQ ID NO: 1) and the PF-KEX2b peptide (SEQ ID NO: 2) comprise serine residues at positions 47 and 77 of the amino acid sequence.

The encoding DNA sequence of the PF-KEX2b peptide was cloned in an E. coli expression vector and the recombinant protein was produced, isolated and purified for use in studies described infra.

### Example 2: Assessment of the Immunogenicity of the PF-KEX2b Peptide

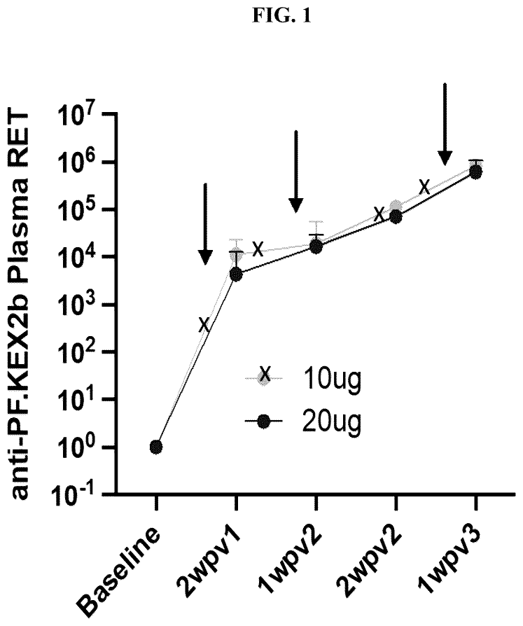

To evaluate the immunogenicity of the PF-KEX2b peptide (SEQ ID NO: 2) as described herein, BALB/c mice (50% male) were immunized (inoculated) intramuscularly (IM) with 10 μg (line containing “x”s on graph) or 20 μg (solid line on graph) of the PF-KEX2b peptide mixed 1:1 with ALHYDROGEL® adjuvant. Twenty-one (21) days following the first immunization, the mice were boosted with the same dose of the PF-KEX2b peptide plus ALHYDROGEL®. A third immunization with the same dose was administered 21 days after the second. The immunization time points and the antibody titer at various time points are shown in FIG. 1. The results of the study demonstrated that immunization of animals with either 10 μg or 20 μg of the PF-KEX2b peptide and ALHYDROGEL® adjuvant generated robust antibody titers after each inoculation (immunization).

### Example 3: Assessment of the Immunogenicity and Protective Efficacy of PF-KEX2b Peptide and ALHYDROGEL® Immunization in a Murine Model of Invasive Aspergillosis Associated with Aspergillus fumigatus (AF) Infection

A mouse model of invasive pulmonary aspergillosis (IPA) was used to assess whether mice immunized with the PF-KEX2b peptide as described herein and adjuvant mounted a humoral immune response and immune antisera containing anti-PF-KEX2b antibodies that protected the immunized animals from subsequent infection by Aspergillus pathogen.

### Vaccine Construction and Purification

A polynucleotide encoding the PF-KEX2b (SEQ ID NO: 2) peptide was cloned into the pET28b (+) expression vector (Novagen) in Escherichia coli BL21(DE3) pLysS (ThermoFisher Scientific) and purified by affinity chromatography.

### Immunization and Immunosuppression

Mice were immunized with 10 or 20 μg PF-KEX2b immunogen prepared 1:1 with adjuvant (e.g., ALHYDROGEL®) according to the adjuvant guidelines, via intramuscular (IM) injection. In some embodiments, e.g., using TiterMax adjuvant, the mice were immunized subcutaneously at the base of the tail. Control mice were sham-immunized with PBS and the same adjuvant. Two weeks following immunization, all mice underwent an immunosuppressive regimen using tacrolimus and hydrocortisone injected subcutaneously (SC). This regimen was administered for six days during which time trimethoprim sulfamethoxazole was added to the drinking water to control secondary infections.

Aspergillus fumigatus Challenge and Monitoring

A. fumigatus Af293 conidia were maintained on solid 1% glucose minimal medium for 72 hours, harvested in 0.01% Tween-20, counted with a hemocytometer, and then were diluted in PBS. Mice were inoculated with 5×10⁶ conidia in 40 μl PBS via intranasal inoculation following the six days of immunosuppression as described above.

Following challenge, mice were monitored twice daily for changes in weight, temperature, and appearance. If weight loss exceeded 20 percent of the baseline body weight or body temperature fell below 29° C., in addition to exhibiting ruffled fur and labored breathing, the animals were humanely sacrificed. At six days following challenge, all remaining animals were humanely sacrificed and their lungs were collected for analysis.

### Fungal Burden

Following sacrifice, the right lungs of all animals were stored in 10% neutral buffered formalin. The fixed lung tissue was embedded in paraffin, cut, and stained with Gomori's modified methanamine silver stain. Images of five distinct fields were photographed and fungal burden was quantified according to the guidelines provided by Stolz et al., 2018, J. Vis. Exp., (133) e57155: 1-8.

### Humoral Immune Response to the PF-KEX2b Peptide

This representative experiment was conducted using the PF-KEX2b peptide as immunogen to evaluate the protective efficacy of the peptide and adjuvant immunization in a murine model of invasive aspergillosis. As such, BALB/c mice (female) were immunized three times intranasally, 21 days apart, with 10 μg of the PF-KEX2b peptide mixed 1:1 with ALHYDROGEL® adjuvant. Control mice were sham-immunized with PBS and ALHYDROGEL®. Two (2) weeks following the third immunization, the mice were immunosuppressed using tacrolimus (1 mg/kg/day i.p.) and hydrocortisone (125 mg/kg every 3 days) and were challenged intranasally with 5×10⁶ of the A. fumigatus conidia. The challenge study design is shown in FIG. 2A. The results of the study showed that mice that were immunized (inoculated) with the PF-KEX2b peptide and ALHYDROGEL® (represented by line containing “x”s on graph in FIG. 2B) had a significant reduction in mortality compared to the sham-immunized control mice (represented by solid line on graph in FIG. 2B), (p=0.0323). As observed in FIG. 2B, immunization of animals with the PF-KEX2b peptide and ALHYDROGEL® significantly reduced Aspergillus-infection-related mortality and invasive aspergillosis in a murine model of immunosuppression.

### Statistical Analyses

All statistical analyses were performed using GraphPad Prism (GraphPad Software, La Jolla, CA). Reduction in IPA-related mortality was assessed using Fisher's exact test. The relative risk in IPA-mortality was determined using Koopman asymptotic score. Differences in fungal burden by GMS staining were analyzed by Mann-Whitney U tests.

### Example 4: Generation of Recombinant Kexin Proteins

The Pan-fungal Kex peptides PF-KEX1b (PF-KEX1b) and PF-KEX2b (PF-KEX2b) were back-translated (www.ebi.ac.uk/Tools/st/emboss_backtranseq/) with an Escherichia coli K-12 codon bias and inserted into the expression vector pET-28b (+) using NcoI and BamHI restriction sites (GenScript). Each insert contained an additional GC 5′ to the Kex sequences followed by CG to keep the Kex sequence inserts in frame. Plasmids were transformed into Escherichia coli BL21 (DE3) cells and plated on LB agar supplemented with 40 μg/mL kanamycin to select for transformed clones. The recombinant PF-KEX1b and PF-KEX2b proteins were expressed and purified as described in Example 5 below.

### Example 5: Procedure for the Purification of Recombinant Kexin Peptides (His-Tagged)

This example describes a protocol for purifying recombinantly produced (pET28b vector), (Millipore-Sigma, US) PF-KEX1b and PF-KEX2b peptides that are histidine (His) tagged.

### Materials and Equipment

A. LB (Lysogeny Broth) growth medium with kanamycin (40 μg/mL), typically in a 1 L volume, pH to 7.5. 10 g NaCl, 5 g Yeast Extract, and 10 g Tryptone Peptone are admixed; the volume is brought to 1 L with distilled/deionized H₂O.
B. 1 M IPTG solution.
C. Extraction buffer: (sterile filtered), 50 mM Sodium Phosphate, 300 mM NaCl, 10 mM imidazole, 6 M Guanidine-HCl, pH 7.4.
D. Wash buffer: (sterile filtered) 50 mM Sodium Phosphate, 300 mM NaCl, 10 mM imidazole pH 7.4.
E. 1 M imidazole solution in 50 mM Sodium Phosphate, 300 mM NaCl, pH 7.4) and 0.2 μm sterile filter (sterile filtered).
F. HISPUR™ Cobalt resin (ThermoFisher #89966)
G. Disposable 5 mL polypropylene column (Thermo P #29922).
H. His-tag protease inhibitor cocktail (PIC) (Sigma P #8849).
I. Bradford Dye (Bio Rad P #500-0006).
J. Bovine Serum Albumin Standard (ThermoFisher #23209).
K. Coomassie Blue stain containing 0.2% Coomassie Blue, 7.5% acetic acid and 50% ethanol.
L. Coomasie Blue destain containing 50% methanol, 10% acetic acid and 40% dH₂O.
M. Acrylamide Bis 30% (Sigma P #1001356385)
N. N,N,N′,N′-Tetramethylethylenediamine (TEMED) (Sigma P #1001434505).
O. Sodium dodecyl sulfate (SDS) (10% stock solution).
P. Ammonium persulfate (APS) (10% stock solution).
Q. 1.5 Tris buffer pH 8.8 (187 g Tris Base into 1 L dH₂O, bring pH to 8.8).
R. 0.5 Tris buffer pH 6.5 (60.5 g Tris Base into 1 L dH₂O, bring pH to 6.5).
S. Spectra™ Multicolor Ladder-Broad range stained (Thermo P #22634).
T. SDS-PAGE sample buffer (4×).
U. Hoefer gel casting system (model SE250).
   The procedure used is as follows:

Culture and induce protein expression in E. coli: Streak Kexin construct on pET28b (+) onto LB KAN agar plate and incubate at 37° C. overnight (O/N) or at RT on the bench top until adequate bacterial growth/colonization is obtained. (Plates can be stored at 4° C. for ˜1 month). Inoculate a single colony into 10 mL liquid LB KAN 40 (10 μL of 40 mg/mL KAN per 10 mL LB), (allowing ˜1:5 liquid to air ratio), and grow at 37° C. overnight with shaking. Following overnight incubation, dilute culture 1:50 into liquid LB KAN 40 (4 mL of overnight culture into 196 mL of fresh medium) and leave at 37° C. on shaker. Grow cultures to an OD₆₀₀ 0.5 and then add 1 mM IPTG to induce expression; leave at 37° C. on shaker for 4-5 hours. Split total volume of culture among five 50 mL Oakridge tubes (˜40 mL culture per tube). For scaling up: can use 250 mL Oakridge tubes for larger volumes. Harvest cells by centrifugation at 6,000×g and 4° C. for 25 minutes (Can use SS-34 or SLA-1500 rotor). Pour off supernatant and freeze cell pellets at −80° C. until time of use. Do not store E. coli pellets for longer than two weeks prior to protein extraction.

Protein purification using Talon metal affinity resin: Thaw pellet on ice and re-suspend cell pellet in 10 mL extraction buffer+200 μL PIC. Incubate at 4° C. for 2 hours minutes on nutator. Centrifuge suspension at 10,000×g and 4° C. for 20 minutes (use SS-34 rotor). Collect supernatant and keep on ice until Talon resin is prepared. Prepare polypropylene elution column by suspending column in the upright position; adding a few drops of wash buffer to a porous disc, then using reverse end of a Pasteur pipette to depress disc evenly to the bottom of the column.

Prepare Talon resin: Resuspend Talon resin by gently shaking and add 3.5 mL of resin to a 15-mL conical tube and spin for 5 minutes at 500×g. Carefully remove ethanol layer without disturbing resin. Add 10 mL of deionized water to wash resin and spin again for 5 minutes at 500×g. Remove supernatant carefully and discard. Equilibrate resin in 10 mL of extraction buffer and spin for 5 minutes at 500×g. Remove supernatant carefully and discard. Batch bind clarified lysate and equilibrated resin by mixing together and nutating for 1 hour at 4° C. Add lysate and resin suspension to the prepared polypropylene elution column. Discard flow through. Wash resin with 15 column volumes of extraction buffer followed by 15 column volumes of wash buffer. Elution of Pan-fungal peptide 2b. Elute in 1.5 mL fractions with increasing imidazole concentration in wash buffer and collect elution fractions.

Elution gradients: Add 1.5 mL of 75 mM imidazole in wash buffer and collect fraction. Add 1.5 mL of 100 mM imidazole in wash buffer and collect fraction. Add 1.5 mL of 125 mM imidazole in wash buffer and collect fraction. Add 1.5 mL of 150 mM imidazole in wash buffer and collect fraction. Add 1.5 mL (×2) of 175 mM imidazole in wash buffer and collect fraction. Add 1.5 mL (×3) of 200 mM imidazole in wash buffer and collect fraction.

Elution of Pneumocystis Kex1, Pan-fungal peptide 2.

Elution of Aspergillus Kex and Cryptococcus Kex (Insoluble proteins): Resuspend resin in 1.5 mL of 1% SDS and transfer into two 1.5 mL tubes. Boil suspension in a heating block 95° C. Centrifuge suspension for 5 minutes at 500×g. Collect supernatant fraction containing denatured protein. Repeat the above four steps three times to recover additional protein Add 20 μL PIC to each fraction of interest and store at 4° C. (Imidazole solutions should be stored on ice prior to use.)

Quantification of protein in elution fractions (Bradford Assay-low concentration standard curve): Remove BSA-100 μg/mL from freezer (4° C.) and thaw on ice. Set up cuvettes for standard curve and add the specified amounts of both the thawed BSA/dH₂O from the below table (Table 1).

TABLE 1

| Final Conc. | dH₂O | BSA-100 μg/mL |
|---|---|---|
| (μg/mL) | (μL) | (μL) |
| 0 | 200 | 0 |
| 0.25 | 197.5 | 2.5 |
| 0.5 | 195 | 5 |
| 1 | 190 | 10 |
| 2 | 180 | 20 |
| 4 | 160 | 40 |
| 8 | 120 | 80 |

Add an additional cuvette for each fraction and dilute samples 1:50 (20 μL sample+180 μL dH₂O). Prepare Bradford dye 1:4 in dH₂O (10 mL dye+30 mL dH₂O) and add 800 μL to each cuvette (final volume 1 mL). Mix cuvettes individually via inversion and incubate at RT for 15 minutes. After incubation, add 200 μL of the 0 μg/mL BSA standard in replicate to wells A1 and A2 of 96 well flat bottom plate followed by the addition 0.25 μg/mL BSA standard to B1 and B2. Continue to add the BSA standard in increasing concentration to the plate in the same order. Once the entirety of the BSA standard is added to the plate, load samples in replicate into the wells immediately below until no rows remain and then proceed to the top row of the next two columns. After all samples are loaded onto the plate read at 595 nm—“Low-conc. Std. Curve.” Record the linear regression (R²) and BSA standard curve values (Data obtained from assays with R²<0.95 should not be used). Raw values for samples represent a 1:50 dilution and should therefore be multiplied by 50 in order to convert back into μg/mL. Once the concentrations of protein have been determined, fractions intended for plate coating (e.g., ELISA/ELISPOT), injection, etc. must be run on a 15% 2 mm SDS-PAGE gel to evaluate purity.

Identification of protein via SDS-PAGE gel Coomassie Blue staining: For each gel, wash 1× glass cover plate, 1× white aluminum backing plate, 2× black plate spacers, and 1× white 10 lane stacking comb with dH₂O. Rinse with 70% ethanol solution. Confirm that all solidified gel residue from previous use is removed before casting. After all materials have air-dried, take the backing plate and lay it flat on the bench top and place a spacer on each side of the plate before sandwiching with the clear glass cover plate. Confirm that the notches of the spacer are properly aligned to the edges of both plates. Loosen all screws on the casting block and slide the sandwiched plates with spacers into the caster. Confirm that all plates and spacer are even and aligned. Leave ˜3 mm of the sandwiched plates protruding from the bottom of the casting block before carefully tightening the screws so as not to crack the plates. Place the casting block into the holder and set the black plastic plugs into the holder. Turn plugs to depress casting block into the black rubber mat of the holder. Confirm that the bottom of the plates is well sealed by the rubber of the holder in order to avoid leaks. Prepare separating/running gel according to the recipe below for a 15%-SDS PAGE gel and add solution to the cavity between aluminum backing plate and glass cover plate. Allow ˜1.5-2 cm of space at the top of the sandwiched plates for stacking gel. Add ˜1 mL of dH₂O to casting block. The gel will begin to polymerize once the APS/TEMED are added to the solution.

| Separating Gel | 15% | |
|---|---|---|
| Acrylamide Bis 30% | 5 | mL |
| Water (dH₂O) | 2.34 | mL |
| 1.5 Tris Buffer pH 8.8 | 2.5 | mL |
| SDS (10%) | 100 | μL |
| APS (10%) | 50 | μL |
| TEMED | 10 | μL |

Once the gel has hardened (approximately 35 minutes), remove the layer of water and prepare the stacking gel solution from the recipe below. Add solution quickly.

| Stacking Gel | 15% | |
|---|---|---|
| Acrylamide Bis 30% | 700 | μL |
| Water (dH₂O) | 3.2 | mL |
| 0.5 Tris Buffer pH 6.5 | 1250 | μL |
| SDS (10%) | 50 | μL |
| APS (10%) | 60 | μL |
| TEMED | 20 | μL |

Immediately place the white 10 lane stacking comb into the stacking gel and allow to fully polymerize (about 10 minutes). Prepare samples to run on gel: (5 μg protein per well). If the fraction concentration is <165 μg/mL, use 22.5 μL of sample+7.5 μL 4× Sample Buffer. If the fraction concentration is >165 μg/mL=>sample vol.=5 μg/(conc. (μg/mL)/1000) & 4× Sample Buffer vol.=(⅓)*sample vol. Heat-inactivate all samples for 10 minutes at 56° C. in the water bath. Remove 15% SDS-PAGE gel from casting block and attach to the running apparatus with 2× red clips. Fill the cavity of the running apparatus and the bottom tray with 1×SDS-PAGE running buffer. The stacking comb can now be removed. Add 10 μL of the Broad Range stained (P #26634) SPECTRA™ Multicolor Ladder to first well of the gel followed by 30 μL of the prepared samples to the subsequent wells. Once all samples are loaded, attach the electrodes to their appropriate terminals and turn on the power supply (red to red, black to black). Allow the gel to run at ˜80-120 volts for 1.5-2.5 hours until the dye band runs of the bottom of the gel. At that point, turn off the machine and disconnect the electrodes (Note: Lower voltages and lower time intervals increase the quality of the resulting gel.). Drain the running buffer from the running apparatus. Remove the red clips, spacers, and gently detach the glass cover plate from the gel casting frame. Use the hard plastic straight edge of the gel scraper to cleave the stacking gel off and into the trash. Divide gel as necessary for further assays, i.e. Western Blot, etc. (it is not necessary to notch a corner of the gel to establish orientation because of the stained ladder used.). For the separating gel that will be stained, wash 3× w/dH₂O for 15 minutes. Add ˜25 mL of Coomassie Blue stain to the gel for 2+ hours or overnight if necessary. Destain with Coomassie Blue de-stain until optimal band color/gel transparency is obtained. Take a picture and save as JPG/TIF file.

### Example 6: PF-KEX2a and PF-KEX2b Peptide Enzyme Linked Immunosorbent Assay (ELISA)

This example describes a protocol for performing an ELISA immunoassay utilizing the pan-fungal peptide 2a (PF-KEX2a) or pan-fungal peptide 2b (PF-KEX2b) peptide antigen used as immunogen in the Examples described supra. The ELISA was conducted to detect (and quantify) the presence of anti-fungal PF-KEX2a or PF-KEX2b antibodies in a sample, e.g., blood, plasma, serum, bronchoalveolar lavage, or biological fluid sample. Antibodies to be detected (and quantified) are directed against, reactive with, and/or bind to, for example, the PF-KEX2b peptide immunogen or the PF-KEX2a peptide immunogen. By way of representative example, antibodies were generated against the PF-KEX2b peptide used as an immunogen as described in Examples 1 and 2 supra and as shown in FIGS. 1, 2A and 2B.

### Materials and Equipment:

PF-KEX2b protein, which may be purified as described in the Examples herein. 1×PBS; Immulon high-binding (4HBX) Flat bottom microtiter plates (Thermo #3855); Blocking buffer: 5% skim milk in 1×PBS; Wash buffer: 1× Phosphate-buffered Saline (PBS)+0.05% Tween-20; Secondary Antibody: Goat anti-human immunoglobulin-conjugated horseradish peroxidase (1:10,000 for IgG; Sigma-Aldrich); Normal human plasma (Atlanta Biologicals, Inc., Lawrenceville, GA). Negative/normal control plasma with undetectable absorbance at OD₄₅₀ (i.e., equal to or less than dilution buffer alone) in KEX-ELISA (Enzyme linked immunosorbent assay) at a dilution of 1:100, used as negative control; Substrate: 3,3′,5,5′-Tetramethylbenzidine (TMB) peroxidase substrate (such as SureBlue TMB substrate, 1-component; KPL, Inc.); Stop solution: 1M H₂SO₄; Adhesive sealing film for microplates (Plate sealers) (such as SealPlate non-sterile films from Excel Scientific, cat #100-SEAL-PLT); 96-well plate reader (any system capable of reading OD at a wavelength of 450 nm). The procedure for performing the ELISA is as follows:

Coating/blocking ELISA plates with PF-KEX2b protein: Prepare PF-KEX2b protein in 1×PBS at 5 μg/mL. Add 50 μL of diluted PF-KEX2b per well of Immulon 4HBX flat-bottom ELISA plates. Cover plates tightly with Parafilm or plate sealers and incubate overnight at 4° C. Following overnight incubation, remove buffer by flicking into sink or bucket and tap plate onto absorbant pad or paper towels to remove excess. Wash plates 2× with wash buffer (PBS 0.05% Tween-20) (approximately 200 μL wash buffer per well for each wash, flicking and tapping plate between washes). Add 100 μL of blocking buffer (5% milk/PBS) to each well and incubate for 1 hour at 37° C. Empty plates, wash 2× with wash buffer. The plates can be sealed and frozen at −20° C. at this step, until ready for use.

Handling of plasma or other infectious fluids (e.g., bronchoalveolar lavage (BAL) fluid supernatant, etc.)—First-time use: Remove plasma aliquot from −80° C. freezer.

Option 1: Heat-inactivate entire aliquot at 56° C. for 30 minutes.

Option 2: If heat inactivation of the plasma sample would be detrimental to other potential uses, thaw sample at 4° C. or on ice. Remove an aliquot (˜100 μL), transfer to a new tube, and heat inactivate (30 minutes, 56° C.). Return the remaining sample to the −80° C. freezer, noting that it has been thawed 1×.

Centrifuge the sample at >10,000×g for 1-2 minutes to pellet aggregates prior to use. To prevent contamination in storage, add ˜0.01 to 0.02% NaN₃. Store sample aliquots for up to 6 months at 4° C. For subsequent assays, no further heat inactivation is needed; however, the sample should be centrifuged briefly prior to each use.

ELISA for endpoint titer determination (plasma): Dilute plasma 1:100 in blocking buffer. Add 50 μL of diluted plasma and make serial 2× (or 4×, if needed) dilutions directly in the plate (final volume in each well should be 50 μL) for generation of endpoint titers. Perform assay in duplicate; set up enough plates for all isotypes of interest, e.g., if there are 10 samples and endpoint titers are to be generated for both IgG and IgM anti-PF-KEX2b antibodies, this would require setting up 4 plates (duplicate plates for both IgG and IgM). Include a negative/normal control on each plate. Cover plates with plate sealers and incubate overnight at 4° C. Empty plate (flicking and tapping), wash 4× w/wash buffer. Add 50 μL of secondary antibody (diluted in block) to each well (see appropriate dilutions under Materials and Equipment above). Incubate 1 hour at 37° C. Empty the plate and wash 6× with wash buffer. Add 100 μL of TMB to each well, protect from light and incubate for 30 minutes at 37° C. Add 25-50 μL of stop solution (1M H₂SO₄) to each well. Read OD of plates (on any standard plate reader) at 450 nm within 20 minutes of adding stop solution.

The majority of healthy adults (both humans and non-human primates) have circulating antibodies to Pneumocystis; therefore, when selecting a control sample to be used for calculating endpoint titers, plasma samples must be screened from healthy donors to determine and obtain an appropriate control. In plasma from an appropriate normal/negative control, the PF-KEX2b OD₄₅₀ at a 1:100 dilution should be not more than 0.1; however, the lower the OD of the normal/negative control plasma, the better the control is. To control for plate-to-plate variability, the same normal/negative control should be used on all plates following the selection of an appropriate normal/negative control.

ELISA for endpoint titer determination (BAL Supernatant): Dilute BAL supernatant 1:100 in normal saline. Determine the urea concentration of the BAL supernatant and corresponding plasma sample using QuantiChrom Urea assay (BioAssay Systems Cat #DIUR-500). Follow instructions on kit insert, diluting plasma 1:10 in distilled water and using BAL supernatant without dilution. Plate plasma samples in the wells of a 96-well plate adding 5 μL of standard (1:10 dilution), blank (distilled water) and sample (1:10 dilution) in duplicates. Plate BAL supernatant in the wells of a 96-well plate adding 50 μL of standard (diluted to 5 mg/dL), blank (distilled water) and sample (undiluted). Add 200 μL working reagent (included in kit) and tap lightly to mix. Incubate plasma plate for 20 minutes at room temperature (RT) and read at OD₅₂₀ on a spectrophotometer. Incubate BAL supernatant plate for 50 minutes at RT and read at OD₄₃₀. Calculate urea concentrations ([urea]) for plasma and BAL supernatant as follows: [urea]=(ODsample−ODblank)/(ODstandard−ODblank)*[standard]. The concentration of standard for plasma will be 50 mg/dL and will be 5 mg/dL for the BAL supernatant. Calculate 1:100 dilution of BAL supernatant as follows: Find the 1:100 dilution factor of bal to plasma (Dilution factor=100/(plasma [urea]/bal [urea])). Calculate volumes for dilution for 500 μl total sample (Volume of sample=500 μL/dilution factor; Volume of saline=500 μL−volume of sample). Add the volume of sample and volume of saline to make 1:100 diluted BAL supernatant sample. Add 50 μL of diluted BAL supernatant and make serial 2× (or 4×, if needed) dilutions directly in the plate with normal saline (final volume in each well should be 50 μL) for generation of endpoint titers. Perform assay in duplicate; set up enough plates for all isotypes of interest, e.g., if there are 10 samples and endpoint titers are to be generated for both IgG and IgM-anti-PF-KEX2b antibodies, this would require setting up 4 plates (duplicate plates for both IgG and IgM). Include a negative/normal control in each plate, as described above. Cover plates with plate sealers and incubate overnight at 4° C. Empty plate (flicking and tapping), wash 4× w/wash buffer. Add 50 μL of secondary antibody (diluted in block, see appropriate dilutions under Materials and Equipment) to each well. Incubate for 1 hour at 37° C. Empty the plate and wash 6× with wash buffer. Add 100 μL of TMB to each well, protect from light and incubate for 30 minutes at 37° C. Add 25-50 μL of stop solution (1M H₂SO₄) to each well. Read OD of plates (on any standard plate reader) at 450 nm within 20 minutes of adding stop solution.

Determining Endpoint Titers: Plot OD readings from each sample (at all dilutions) in Excel, or similar program, as a line graph. For the normal/negative control sample, add 0.025 to each value prior to plotting as described below. The endpoint titer is defined by the dilution at which the test sample gives the same OD reading as that of the negative control (i.e., where the lines meet). Generally, the reciprocal endpoint titer is reported; thus, if the dilution is 1:1600, the endpoint titer is reported as 1600. Calculate endpoint titers from each of the duplicate plates, to confirm that the results are consistent between plates. Acceptable error is within one dilution. If reciprocal endpoint titers (RET) from duplicate plates fall within one dilution, average the titers (e.g., when doubling-dilutions are made, and a sample from plate 1 has a RET of 1600 and the RET from plate 2 is 3200, then the average titer is 2400). If endpoint titers on duplicate plates do not fall within one dilution of each other, repeat the ELISA on one additional plate and average the 2 values which are closest.

### Example 7: Procedure for the Purification of Recombinant Kexin 2a and 2b Peptides (Non-His-Tagged)

This example described the purification of the PF.KEX2a (or PF.KEX2b) peptide expressed from the pET-28b+ vector in BL21(DE3) pLys E. coli cells. If PF.KEX2a (pET-28B(+)) was expressed in BLR (DE3) pLys cells, half the volume of cell culture was used for each step in the same buffer volumes. For anion exchange chromatograpy, the elution gradient is 0-2M NaCl instead of 0-1M NaCl.

### Materials and Equipment:

Animal Free Luria-Bertani (LB) Agar w/Kanamycin at 40 μg/ml+Chloramphenicol at 34 μg/mmL; BBL Select APS LB Broth Base 500 g. BD #292438 (Dissolve 10 g of powder+7.5 g Bacto Agar BD #214010 in 500 mL purified water. Mix thoroughly. Autoclave at 121° C. for 15 minutes. Cool until tepid.). Chloramphenicol 1000× Stock [34 μg/ml]. (Sigma #C1919-5G, Dissolve powder in 100% Ethanol, 0.2 μm sterile filter, store at −20° C.). Kanamycin 1000× stock [40 mg/ml]. (Dissolve powder in water, 0.2 μm sterile filter, store at −20° C.). Add 500 μl Chloramphenicol 1000× Stock [34 mg/ml], 500 μl Kanamycin 1000× Stock [40 mg/ml] to 500 mL tepid LB Agar. Final Concentration Chloramphenicol [34 μg/ml], Kanamycin [40 μg/ml]. Pour ˜25 plates every 6 months as needed.

Animal Free Luria-Bertani (LB) Broth w/Kanamycin at 40 μg/ml+Chloramphenicol at 34 μg/mmL; BBL Select APS LB Broth Base 500 g. BD #292438. (Dissolve 40 g of powder in 2 L purified water. Mix thoroughly. Autoclave at 121° C. for 15 minutes). Add 1 mL Chloramphenicol 1000× Stock [34 mg/ml], 1 mL Kanamycin 1000× Stock [40 mg/ml] to 1 L LB. Final Concentration Chloramphenicol [34 μg/ml], Kanamycin [40 μg/ml]. Prepare a total of 4 L LB broth+KAN/Cloro for every purification. Kimble 20×150 mm Borosilicate Glass Culture Tubes Duran Wheaton Kimble #7500-20150—for O/N culture. 4×2000 mL Erlenmeyer Flasks for subculture. 1M IPTG solution (0.22 μm sterile filter, 200 μl aliquots). Oakridge tubes (4×500 ml Beckman Catalog #361691; 4×50 ml Beckman Catalog #357003). Centrifuges: (Beckman Coulter Avanti J-E High Speed Centrifuge (JLA-10.500 Fixed Angle Rotor (Max 10000 RPM; 18,600 g). (JA-25.50 Fixed Angle Rotor (Max 25000 RPM; 75,600 g). Labnet Prism-R Refrigerated Microcentrifuge. Prepare 500 ml 20 mM Tris HCl, pH8.0, (10 mL 1M Tris HCl, pH 8.0). Fill to 500 ml with dH₂O. 0.22 μm sterile filter with 250 mL, PES Membrane (Genesee Scientific #25-227); Store at RT. CellLytic B Cell Lysis Reagent (Sigma #B7435)-500 ML, Store at RT. Benzonase (5000×), (Sigma #E1014-25KU), Store at −20° C. Protease inhibitor cocktail (PIC) (Sigma P #8849).
A. Millex-GP Syringe Filter Unit, 0.22 μm, polyethersulfone, 33 mm, gamma sterilized. Fast Flow & Low Binding Millipore Express PES Membrane. Millipore #SLGPR33RS (Replacement for Millipore #SLGP033RS).
B. BD 60 mL Syringe. LUER-LOK™ Tip. (BD #309653).
C. Anion Exchange Chromatography Buffer A (Running Buffer): Prepare 500 ml 6M Urea, 20 mM Tris HCl, pH 8.0 (Reagents: 1M Tris-HCl, pH 8.0; (Quality Biological Catalog #351-007-101); Urea: MW=60.06 g/mol).

Procedure: Weigh out 180.18 g Urea; add 10 mL 1M Tris-HCl, pH 8.0; dissolve mixture in ˜400 ml dH₂O and stir with stir bar; sonicate in water bath at 30° C. for ˜10 minutes after stirring; fill to 500 ml with dH₂O; filter sterile with 0.22 μm 500 mL, PES Membrane, Genesee Scientific #25-225; prepare fresh on the day of performing ion exchange chromatography. Store at 4° C. to prevent urea decomposition and carbamylation of proteins.
D. Anion Exchange Chromatography Buffer B (Elution Buffer): Prepare 250 ml 6M Urea, 1M NaCl, 20 mM Tris HCl, pH 8.0. (Reagents: 1M Tris-HCl, pH 8.0, Quality Biological Catalog #351-007-101; NaCl: MW=58.44 g/mol; Urea: MW=60.06 g/mol).

Procedure Weigh out 90.09 g Urea; weigh out 14.61 g NaCl; add 5 mL 1M Tris-HCl, pH8.0; dissolve mixture in ˜200 ml dH₂O and stir with stir bar; sonicate in water bath at 30° C. for ˜10 minutes after stirring; fill to 250 ml with dH₂O; filter sterile with 0.22 μm 250 mL, PES Membrane, Genesee Scientific #25-225; prepare fresh day on the day of performing ion exchange chromatography. Store at 4° C. to prevent urea decomposition and carbamylation of proteins.

FPLC Solutions for Column Storage. Sterile filter all solutions. 20% Ethanol, 0.2 μm sterile filter. For AKTA cleaning and column storage buffer and dH₂O.

FPLC Solutions for Cleaning HiTrap Capto Q column. Sterile filter all solutions. 2M NaCl and 1M NaOH.

Anion Exchange Column: HiTrap Capto Q 5×5 mL GE Healthcare Life Sciences/Cytiva (Catalog #11001303); Store column at 4° C. in 20% Ethanol.

Size Exclusion Chromatography Column: Superdex 75 Increase 10/30 GL; (GE Healthcare Life Sciences/Cytivia Life Sciences Catalog #29148721); Store column at 4° C. in 20% Ethanol.

Size Exclusion Chromatography Buffer: Prepare 500 mL 6M Urea, 250 mM NaCl, 20 mM Tris-HCl, pH 8.0. Reagents: 1M Tris-HCl, pH8.0 (Quality Biological Catalog #351-007-101), NaCl (MW=58.44 g/mol); Urea (MW-60.06 g/mol)

Procedure Weigh out 180.18 g Urea; Weigh out 7.305 g NaCl; Add 10 mL 1M Tris-HCl, pH 8.0. Dissolve mixture in ˜400 ml dH₂O and stir with stir bar. Sonicate at 30° C. for ˜10 minutes after stirring. Fill to 500 ml with dH₂O. Filter sterile with 0.22 μm 500 mL, PES Membrane (Genesee Scientific #25-225). Prepare fresh on the day of performing ion exchange chromatography. Store at 4° C. to prevent urea decomposition and carbamylation of proteins. Fast protein liquid chromatography (FPLC) System: Trent Lab FPLC or CVI Protein Core. Superloop 50 mL (GE Biosciences/Cytivia #18111382). AMICON® Ultra-4 Centrifugal Filter Unit, Ultracel-3 regenerated cellulose membrane, 4 mL sample volume. (SigmaMillipore #UFC800324). Millex-GP Syringe Filter Unit, 0.22 μm, polyesthersulfone (PES) membrane, 33 mm, gamma sterilized. SigmaMillipore #SLGP033RS. Pierce Detergent Compatible Bradford Assay Kit (ThermoFisher #23246). Western blotting Reagents. Target: PF-KEX2a.

1×SDS PAGE Running Buffer: 25 mM Tris, 192 mM Glycine, 0.1% SDS, pH 8.3; 6× SDS Loading Dye: 12% SDS, 47% glycerol, 60 mM Tris HCl pH6.8, 0.06% bromphenol blue, 5% β. To prepare 10 mL: 1.2 g SDS, 6 mg bromphenol blue, 4.7 ml glycerol, 1.2 ml 0.5M Tris HCl pH 6.8, 2.5mlH₂O. Heat at 37° C. to dissolve SDS in glycerol, Tris buffer, and water. Prepare 950 μl aliquots of loading buffer and store at −20° C. Add 50 μl 14.3M pure β-mercaptoethanol (βME) before use. Final 2βME concentration should be 5% before use. Membrane: iBlot Transfer Stack Nitrocellulose (0.2 uM) ThermoFisher #IB401001. 1° Antibody: High titer sera from monkey #5617 1wpv2 Apr. 3, 2019 vaccinated with PFKEX-his; RET=1,152,000; stored at −4° C. 2° Antibody: Goat anti-monkey IgG (H+L)-HRP [1 mg/ml], (ThermoFisher #PA1-84631). Blocking Buffer: 5% Nonfat dry milk in PBS-T (0.05% Tween-20) or TBS-T (0.05% Tween-20). Prepare at least 1 hr prior to blocking. Wash buffer: PBS or TBS. Standard lab protocols. SuperSignal West Pico PLUS Chemiluminescent Substrate (ThermoFisher #34577). PRECISION PLUS PROTEIN™ Dual Xtra Prestained Protein Standards (#1610377). Ponceau S Staining Buffer (0.2% Ponceau S, 5% glacial acetic acid). Coomassie Stain: Coomassie Brilliant Blue R-250, (Fisher #BP101-25). Destain Buffer (Per Liter: 10% Acetic Acid (100 mL), 40% Methanol (400 mL), 50% dH₂O (500 mL)). Mix 1.25 g Coomassie Brilliant Blue R-250 with 500 mL Destain Buffer. Stir O/N to dissolve.)

When purifying protein from BL21(DE3) pLys cells, prepare 2×4 L cell culture prior to purification; prepare protein purification worksheet for every 4 L cell culture. For each anion ion exchange run, purify protein from 4 L cell culture. For size exclusion chromatography, pool best fractions from 2×4 L anion exchange chromatography runs. Up to 20 mg protein over the size exclusion chromatography column can be injected; therefore, to purify protein from anion exchange chromatography-enriched fractions obtained from 2×4 L cell culture, 2-3 size exclusion chromatography runs will need to be performed.

Culture and induce protein in 2×4 L=8 L total E. coli. Because it is difficult to grow all 8 L in one day, the culture is separated into 2×4 L cell culture lots.

Growing a 4 L Cell culture: Streak out Tagless PFKEX on pET28b in BL21(DE3) pLyS onto LB agar+KAN/Chloro plate and incubate at 37° C. O/N and select for single colonies. Inoculate 8 single colonies into 8×10 mL LB Broth+KAN/Chlor (allowing ˜1:5 liquid to air ratio) in Kimble 20×150 mm Borosilicate Glass Culture Tubes Duran Wheaton Kimble #7500-20150 at 37° C. O/N shaking at 225 rpm. To grow 4 L liquid culture: Following overnight incubation, dilute culture 1:50 into liquid LB Broth+KAN/Chlor. Add 10 mL culture to 500 ml. Repeat for 8×500 ml culture. Culture each 500 ml volume in a 2 L Erlenmeyer flask. Shake for ˜2 hrs at 37° C. or until OD₆₀₀=0.5. Induce protein expression at 0.5 mM IPTG by adding 250 μl 1M IPTG to each ˜500 mL culture. Shake for 4 hrs at 37° C. Harvest cells in 500 mL Oakridge tubes and centrifuge at 6,000×g and 4° C. for 20 minutes in Beckman Avanti J-E centrifuge (JLA-10.500 fixed angle rotor). Discard supernatant. Resuspend each pellet with ˜50 mL 20 mM Tris HCl, pH 8.0 to wash out residual media. Combine all pellets into a single Oakridge tube containing cell paste from 4 L cell culture. Centrifuge at 6,000×g and 4° C. for 20 minutes. Discard supernatant. Remove as much residual liquid as possible to prevent dilution of CelLytic B at time of cell lysis. Store cell pellets at −80° C. until time of use. Repeat E. coli culture and induction for another 4 L of cell culture.

Isolation of Inclusion Bodies from 4 L of cell culture: Thaw pelleted cells on ice. Resuspend cell pellets from 4 L of cell culture in 40 mL CelLytic B Cell Lysis Reagent+8 μL Benzonase+400 μl protease inhibitor cocktail (PIC), (Sigma P8849). Vortex cell suspension 1-2 minutes until thoroughly suspended and mutate cells for 10 min at RT. Centrifuge at 15,000 g for 10 min at 4° C. in 50 mL Oakridge tube. Discard cell lysis supernatant. Lyse cells a second time using a total of 20 mL CelLytic B Lysis for 4 L cell culture. Vigorously pipette and vortex the cell pellet to facilitate cell lysis and break up of pellet material. Centrifuge at 15,000 g for 10 min at 4° C. Prepare 60 mL Inclusion Body Wash by diluting 6 mL CelLytic B+54 mL dH₂O. Wash pellet with 20 mL of prepared Inclusion Body Wash. Vigorously pipette and vortex the resuspended material to facilitate cell lysis and inclusion body isolation. Centrifuge at 15,000 g for 10 min at 4° C. Repeat 2×. Wash pellet with 20 mL of 20 mM Tris HCl, pH 8.0. Vigorously pipette and vortex the resuspended material to facilitate cell lysis and inclusion body isolation. Centrifuge at 15,000 g for 10 min at 4° C. Discard supernatant. Store purified inclusion bodies at −80° C. until day of anion exchange chromatography. Repeat for second 4 L cell culture pellet.

Solublization and Extraction of His-Tag Free PF-KEX peptide from Inclusion Bodies: Use 4 L cell culture for each anion exchange chromatography run. Thaw purified inclusion bodies on ice. Resuspend pellets in 50 mL “Buffer A” (Running Buffer): 6M Urea, 20 mM Tris HCl, pH8.0 plus 200 μl protease inhibitor cocktail in a 50 mL Oakridge tube. Vigorously pipette and vortex the resuspended material to break up inclusion bodies and encourage protein extraction. Sonicate in water bath at 30° C. for 10 min if needed to dissolve pellet. Nutate suspension for 2 hrs at RT. Place suspension (in 50 ml conical tube) on ice and store O/N at 4° C. to crash out undesired non-Kexin proteins, (e.g., to prevent Capto Q column from reaching pressure limit. Non-Kexin proteins will crash out on column at 4 C). The next morning, centrifuge at 15,000 g for 15 min at 4° C. Harvest supernatant and record supernatant volume. Save 100 μl suspension prior to filtration (label “pre-filter) to determine loss due to filtration. Save some of the pellet to analyze extraction efficiency (label “Urea Pellet”). Load sample/supernatant in BD 60 ml Syringe. LUER-LOK™ Tip. BD #309653, and connect syringe to Millex-GP Syringe Filter Unit, 0.22 μm, Millipore #SLGPR33RS. Syringe filter sample before proceeding to anion exchange chromatography to eliminate particulates and de-gas sample. Save 100 μl of suspension prior to anion exchange (label “Post-filter/AIEX starting material) to estimate amount of protein prior to anion exchange chromatography. Store filtered sample on ice prior to anion exchange chromatography.

Anion Exchange Chromatography (AIEX): Use FPLC equipped with 50 mL Superloop: One anion exchange run was performed for every 4 L cell culture. Protocol: Open Unicorn Software Program. Hook up HiTrap Capto Q column. Do not introduce air bubbles into the column. Wash out 20% Ethanol storage solution from column with 5 mL dH₂O. Swap out storage buffers to AIEX Buffers (Buffer A: 6M Urea, 20 mM Tris-HCl, pH8.0; Buffer B: 6M Urea, 1M NaCl, 20 mM Tris-HCl, pH8.0). Equilibrate column with 5CV/5 mL Buffer A→5CV/5 m Buffer B→5CV/5 mL Buffer A. Inject sample without air. Approximate injection amount to input in the computer. Document the injection volume to estimate purification efficiency. Run Program Conditions: Bind ˜sample volume to column. Collect ˜sample volume flow through. Save 100 μl of “Flow thru” for analysis. Wash with 100 mL/20CV of Buffer A. Elute in 25 mL/5CV in a 0-100% gradient of Buffer B. (Fraction Size=1.0 mL; Flow Rate: 0.8 mL/min; Pressure Limit: 0.5 MPa). Wash column with 75 ml/15CV Buffer B. Wash with 25 ml/5CV Buffer A.

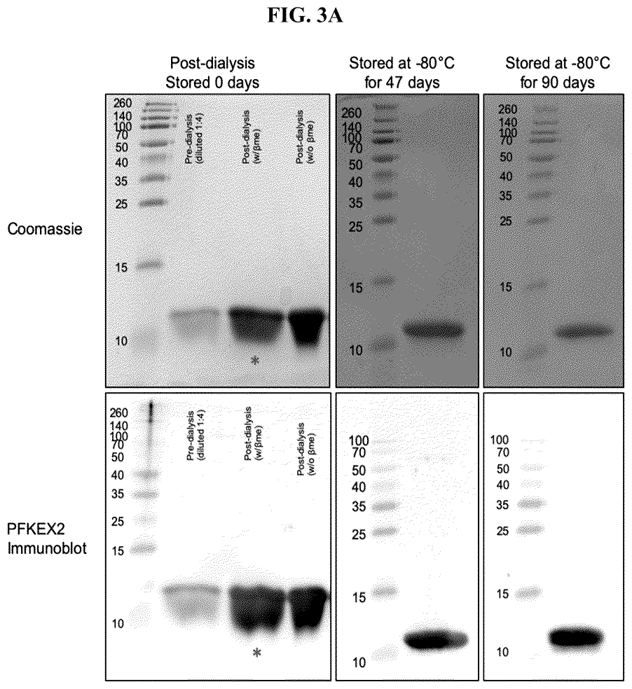

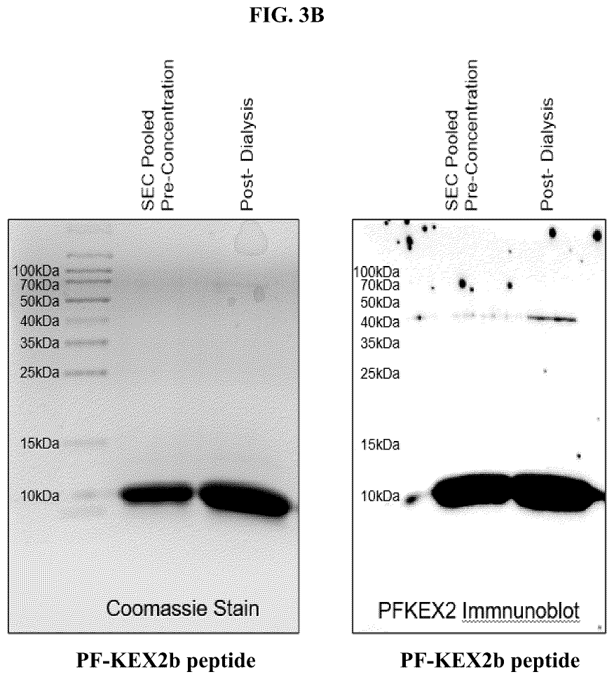

To re-use column (to be repeated after every run): Wash with at least 2CV of 2M NaCl. Wash with at least 4CV of 1M NaOH. Wash with at least 2CV of 2M NaCl. Rinse with at least 5CV of dH₂O. If repeating AIEX run, equilibrate column with 25 ml/5CV Buffer A. Following the last stripping/clean in place protocol, wash with 5CV 20% ethanol and store column in 20% ethanol at 4° C. After sample run collect the “Flow thru”, “Wash”, and elution fractions. Print out post-run chromatogram. Store samples at 4° C. for up to 1 week while performing Bradford Assay and SDS-PAGE analysis. For long-term storage, store at −80° C. FIGS. 3A and 3B provide Coommassie stained gels and immunoblot analyses showing the purified PF-KEX2a peptide (FIG. 3A) and the PF-KEX2b peptide (FIG. 3B) (post-dialysis). FIG. 3A demonstrates the stability of the purified PF-KEX2a peptide over time, e.g., 47 and 90 days stored at −80° C.; similar results are expected for the PF-KEX2b peptide.

Quality Control Analysis Post-Anion Exchange Chromatography/Ion Exchange Chromatography (IEX): Up to 30 samples are typically analyzed by SDS-PAGE (Western Blot & Coomassie Staining) from each anion exchange chromatography run. As many anion exchange chromatography fractions with the highest concentrations of protein that will fit on each gel for the highest levels of protein recovery are analyzed. Include the analysis of control samples as they are necessary to calculate the purification efficiency. Measure all protein fractions by Bradford assay using kits that are compatible with 6M Urea buffers and follow the enclosed protocols. For example, Pierce Detergent Compatible Bradford Assay Kit Protocol (Thermo Fisher Catalog #23246) and BioRad Protein Assay (Catalog #500-0006); the linear range of this microtiter assay is 0.05-0.5 mg/ml.

Measure elution fractions and standards in duplicate. Include the following collected samples for QC analysis: Urea Pellet: (Use this to check extraction and solubilization efficiencies. Boil a small volume 1:1 in ˜100 μl 1% SDS for 5 min. Centrifuge at 16,000×g for 5 min. Collect supernatant for analysis SDS-PAGE analysis. Pre-filter sample: Confirm amount of protein was collected in the supernatant after extraction. Use the sample volume before filtering to calculate the yield. Post-filter/AIEX starting material: Determine the amount of protein was injected through the column. Use the sample volume recorded above to calculate the protein amount injected over the column. Flow-thru: Used to check efficiency of anion exchange column binding. Wash. Determine if any precipitate fell out of solution (i.e., the appearance of peaks in stripping or otherwise). Anion exchange fractions: Read samples at 595 nm. Extrapolate fraction concentrations from a standard curve and calculate the protein yield from anion exchange chromatography.

Dilute samples with water and add 6× Laemmli Buffer. Coomassie staining (10 μg/well or up to 30 μl). Western Blotting (5 μg/well or up to 30 μl). Add 6 μl of 6× Laemmli Buffer (5% 2-mercaptoethanol) to 30 μl diluted sample. Centrifuge at full speed for 5 minutes to pellet debris. To avoid potential protein modification problems, samples containing urea were not boiled. Store prepared samples at −80° C. until next day if necessary.

Gel Electrophoresis: Prepare 15% resolving/4% stacking polyacrylamide gels according to the manufacturer's protocol. (Handcasting Polyacrylamide Gels; Bio-Rad Laboratories, Inc., Bulletin 6201). Use 15 well combs. Assemble running apparatus and fill inner chamber and bottom tray with 1×SDS-PAGE running buffer. Remove comb and wash out wells. Do not re-use inner chamber buffer. Load 10 μL of the Broad Range stained (P #26634) SPECTRA™ Multicolor Ladder diluted in 1× Laemmli buffer. Gels will run straighter if all samples/standard are the same volume and all wells are full. Load samples. Run the gel at constant voltage (˜80-120 volts) for 1.5-2.5 hrs until the dye front runs off the bottom of the gel. Once the gel is finished running, remove stacking gel with scraper. Wash gels with dH₂O before Coomassie Staining or Western Blotting (see Western Blotting Protocol).

Membrane transfer: Assemble Invitrogen IBLOT™ Gel Transfer System. Open the lid of the device and place anode stack w/red plastic bottom directly on the blotting surface, aligned to the right Gel barrier. Place pre-run 15% SDS Page gel on the transfer membrane of the anode stack. Wet IBLOT™ filter paper with dH₂O and place on top of the gel, making sure to remove air bubbles with the blotting roller. Remove the sealing of the cathode stack and place gel side down on top of the filer paper. Remove all air bubbles with the roller. Attach a removable sponge with the metal contact on the upper right corner of the lid. Close the lid, secure the latch, and turn on the power switch (located on back of the machine). Select program #3 (P4) ˜20 volts and adjust the default time from 5 minutes. Press the Start/Stop button and wait for the light to turn from red to green. Once the gel run time has elapsed, the machine will beep and the Start/Stop button should be pressed again until the light turns from green to red. At this point turn off the power switch and disconnect the cord. Un-clasp the lid and make sure that the stained ladder has transferred to the nitrocellulose membrane appropriately. Trim the membrane to the desired size with a scalpel and use forceps to transfer it to a light resistant tray.

Ponceau Staining:—To confirm protein has been transferred, wash membrane with dH₂O. Add ˜1 mL of ponceau stain to the membrane and nutate by hand for ˜1 min. Remove ponceau stain and rinse membrane with dH₂O until the double-banded mini kexin protein becomes visible (mini kexin˜10-12 kDa). Image membrane and save as a TIFF. Continue to rinse stain with dH₂O until all the stain is removed.

0.025% Coomassie Staining to measure purity of fractions: Add ˜25 mL of Coomassie Blue stain. Microwave for 2×15 sec with lid. Cool by rocking for 15 min at RT. Discard stain and wash with dH₂O until water is clear. Add ˜25 ml of Destain to gel and a Kim Wipe. Rock on nutator O/N at RT. Kim Wipe will absorb excess Coomassie stain so that destain will not need to be changed overnight. Image gels and save as TIFF files.

Western blotting: Membrane: Nitrocellulose (0.2 μM), (ThermoFisher #IB401001). 1° Antibody: High titer sera from monkey #5617 1wpv2 vaccinated with PFKEX-his; RET=1,152,000; stored at −4° C. 2° Antibody: Goat anti-monkey IgG (H+L)-HRP [1 mg/ml], (ThermoFisher #PA1-84631). Blocking Buffer: 5% Nonfat dry milk in PBS-T (0.05% Tween-20) or TBS-T (0.05% Tween-20). Prepare at least 1 hr prior to blocking. Block for 1 hr at RT or O/N at 4° C. in 10 mL blocking buffer. Add primary (1:5,000-10,000) in blocking buffer without washing (1-2 μl primary+10 mL blocking buffer). Incubate 1-2 hr at RT or O/N at 4° C. Wash 3×TBS-T (0.05% Tween-20)-15 min, 10 min, and 5 min. Add secondary (1:10,000) in blocking 1 hr at RT. (Add 1secondary+10 mL blocking buffer). Wash 3× in TBS-T. Develop with PicoWest. Image membranes and save as TIFF files.

Following Anion Exchange Chromatography: Pool the best fractions from both anion exchange chromatography runs for size exclusion chromatography. Do not proceed with size exclusion chromatography with poorly enriched samples. The fractions should look relatively clean, otherwise the protein will crash out on the column and clog it. If the protein crashes out, and an increase in column pressure is observed, the size exclusion chromatography column will require deep cleaning with pepsin digestion. The size exclusion chromatography column can bind a maximum of 20 mg per SEC run. The maximum loop size for size exclusion chromatography is 500 μl. The more concentrated the sample, the sharper the chromatogram peaks. However, the size exclusion chromatography column can only hold up to 20 mg protein for each 500 μl sample injected; therefore, the concentrated protein needs to be ≤20 mg in 500 μl. If the pooled samples contain >20 mg, multiple size exclusion chromatography runs need to be performed and scaled accordingly. Concentrate the protein in discrete volumes of 500 μl intervals for discrete numbers of runs. To perform an SEC run the amount of pooled protein is less than 20 mg, concentrate the protein into a volume of 500 μl. Perform 2 SEC runs if the pooled protein concentration is between 20-40 mg. Concentrate protein as above into 1000 μl. Perform 3 SEC runs if the pooled protein concentration is between 40-60 mg of pooled protein. Concentrate protein into 1500 μl.

Protein Concentration with Amicon® Ultra-15 Centrifugal Filter Unit (SigmaMillipore #UFC900324): Pool fractions with highest purity and concentration of protein following anion exchange chromatography and record the volume of the pooled protein. Save 100 μl of this pooled volume for downstream analysis and label as “AIEX Pooled” to calculate approximate yield of protein from pooled samples. Prepare Amicon® Ultra-15 Centrifugal Filter Unit (SigmaMillipore #UFC900324) by equilibrating filter by filling the column to the maximum volume with diH₂O for 5 min to remove residual storage glycerol on the column and prevent protein loss. Centrifuge at 3,000 g for 5 min at RT. Remove water from top and bottom of spin column. Add pooled volume over Amicon® Ultra-15 Centrifugal Filter Unit (SigmaMillipore #UFC900324). Centrifuge at 3,000 g for 60 minutes at RT. Centrifuge until the desired estimated protein concentration and volume are attained.

Size Exclusion Chromatography (SEC): Connect Superdex 75 Increase 10/300 GL column to FPLC. Outfit FPLC with a 500 μl loop. Wash out 20% ethanol storage solution with 1.5CV/36 ml dH₂O. Equilibrate column in 1.5CV/36 mL size exclusion chromatography buffer (6M Urea, 250 mM NaCl, 20 mM Tris-HCl, pH 8.0). The SEC buffer must contain NaCl to maintain protein solubility during the SEC run to avoid protein crashing out. Flush the 500 μl loop with SEC buffer to confirm that there are no leaks. Inject sample, typically ˜500 μl. Run the SEC Program: Inject: 500 μl (Elution Volume: 1.5CV/36 ml). Collect 1.0 ml fraction (Flow Rate: 0.8 mL/min.; Pressure Limit: 5.0 MPa). Collect fractions and chromatogram. Typically fractions will elute by 15 ml. When all runs have been completed, wash the column with 1.5CV/36 ml H₂O. Wash with 1.5CV/0.5M NaOH. Wash with 2CV/48 mL H₂O. Wash column with 2CV/48 mL 20% ethanol. Store column in 20% ethanol. Perform deep/rigorous cleaning if the column exceeds pressure limits: Wash with 1.5CV/36 ml H₂O. Fill column with a solution of 1 mg/ml pepsin in 0.1M acetic acid containing 0.5M NaCl and leave overnight at RT to digest contaminants off column. Wash with 2CV/48 ml H₂O. Wash column in 2CV/48 mL 20% ethanol. Store column in 20% ethanol.

Quality Control Analysis Post-Size Exclusion Chromatography (SEC): Typically, up to ˜15 samples are analyzed by SDS-PAGE (Western Blot and Coumassie Staining) from each size exclusion chromatography run, as 1×15 well SDS-PAGE gels are used for each analysis parameter and the gel will accommodate up to 14 samples, excluding the molecular weight ladder. The analysis includes as many size exclusion chromatography fractions with the highest concentrations of protein that will fit on each gel that corresponds to the major peaks on the chromatogram. Analyze the “AIEX Pooled Sample” to allow for calculation of the purification efficiency of the SEC polishing step. The following parameters are calculated: the amount of protein in AIEX pooled sample fractions; the total protein in fractions recovered; the yield of purified protein recovered from clean fractions based on the Coomassie and Western blot analysis (% recovery=best SEC pooled fractions/AIEX pool×100); the yield of purified protein recovered from 8 L cell culture. i.e. X mg protein/L cell culture.

To analyze protein purity and specificity, size exclusion chromatography (SEC) protein fractions were electrophoresced on a gel and stained with Coomassie blue dye. Western blots were also performed on the samples. By way of example, each lane of the protein gel was loaded with 5 μg of protein, e.g., the PF-KEX2a peptide. In an experiment, the yield of purified protein from combined PF-KEX2a lots was found to be 21 mg/8 L of cell culture, or 2.63 mg/L of cell culture before dialysis.

Urea removal (for in vivo use only): The following protocol relates to the dialysis of proteins for in vivo use. When performing buffer exchange through diafiltration, ensure that PF.KEX2a protein does not precipitate out (crash out) of solution. Its solubility in 1×PBS is estimated to be between 1.5-2 mg/ml after dialysis only. If dialyzing protein for in vivo use, pool fractions≥2 mg/ml. Pool a minimum of 3 mL of purified protein to fill a single dialysis cassette. Dialysis must be performed in discrete intervals of 3 mL. % Recovery is less than 50% if the cassette is not full or the protein concentration is less than 1.5 mg/ml. Typical % recovery is between 37-63.4%. Pool the remaining less concentrated fractions for use in in vitro assays (e.g., ELISA and ELISPOT assays). If using protein to coat plates for ELISA/ELISPOT protein will maintain solubility if diluted from urea into 1×PBS or sterile water. Pool the fractions with the highest purity and concentrations of protein (≥1.5 mg/ml) following size exclusion chromatography. Calculate approximate yield of protein from pooled samples. Save 50 μl of the pooled volume for downstream analysis and label as “SEC Pooled Pre-concentration.” Perform Dialysis to remove urea. Dialysis promotes maximum solubility compared to diafiltration or TCA precipitation.

Dialysis (Pur-A-Lyzer Maxi 3500 Dialysis Kit. Sigma #PURX35005-1KT): Prepare 4 L of 1×PBS pH 7.4. pH the buffer for in vivo use. Fill the Pur-A-Lyzer with 2-3 mL of ultrapure water and incubate for at least 5 minutes. Empty the tube. Check carefully that there is no water leaking from the tube, as absorption of water by the membrane will cause a decrease in water level. Load the sample into the Pur-A-Lyzer tube. Close the tube with the provided caps (do not apply force). The sample volume should be in the range of 0.1-3 mL. If a small volume is used, load the sample close to the inner membrane. Set up apparatus in cold room ˜4° C. Place the loaded Pur-A-Lyzer tube in the supplied floating rack and then place the rack in a stirred beaker containing 2 L of 1×PBS pH 7.4. The floating rack can hold 1-7 Pur-A-Lyzer tubes. Adjust the stir bar speed. Dialyze for ˜2 hrs. Change the dialysis buffer with 2 L fresh 1×PBS pH 7.4. Dialyze O/N at 4° C. with stirring. Remove dialysis cassette from dialyzing solution promptly the next morning. Pipette the sample carefully from the Pur-A-Lyzer to a clean tube. Analyze the dialyzed samples prior to and following analysis by Bradford assay, Coomassie staining, and Western blot analysis as described above. Perform endotoxin testing if necessary using Pierce Chromagenic Endotoxin Quanti Kit (Thermo Scientific #A39552). If using pooled/concentrated protein for further use, e.g., immunization/vaccination, perform a Bradford assay to quantify protein and repeat the Coomassie and Western blot assays to analyze the final product for potential protein degradation. Record endotoxin values.

### OTHER EMBODIMENTS

From the foregoing description, it will be apparent that variations and modifications may be made to the aspects and embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other of the disclosed embodiments or portions thereof.

All patents and publications mentioned in this specification are herein incorporated by reference in their entireties and to the same extent as if each independent patent and publication were specifically and individually indicated to be incorporated by reference.

What is claimed is:

1. An immunogenic peptide or a polynucleotide encoding an immunogenic peptide comprising, consisting, having at least 98% sequence identity to one of the following amino acid sequences:
DDDGKTVDGPSPLVLRAFINGVNNGRNGLGSIYVFASGNGGIYEDNSNFD GYANSVFTITIGGIDKHGKRPKYSEASSSQLAVTYAGGSA (PF-KEX1b) as set forth in SEQ ID NO: 1, or an immunogenic fragment thereof; or
PDDGKTMEGPDILVLRAFINGVQNGRDGKGSIYVFASGNGGGFEDNSNFD GYTNSIYSITVGAIDRKGLHPSYSEASSAQLVVTYSSGSG (PF-KEX2b), as set forth in SEQ ID NO: 2, or an immunogenic fragment thereof;

2. The immunogenic peptide or a polynucleotide of claim 1, wherein the peptide contains a serine(S) amino acid residue at least at positions 47 and 77 of the amino acid sequence.

3. The immunogenic peptide of claim 1, wherein the peptide is recombinant, recombinantly produced, and/or isolated.

4. An immunogenic composition comprising an effective amount of the immunogenic peptide or a polynucleotide encoding the immunogenic peptide of claim 1 and a pharmaceutically acceptable carrier, vehicle, or excipient.

5. A method of eliciting an immune response in a subject, the method comprising administering to the subject the immunogenic peptide or a polynucleotide encoding the immunogenic peptide of claim 1.

6. The method of claim 5, wherein the method is effective to treat or protect the subject against a fungal disease or a symptom thereof.

7. A method of treating or protecting a subject against disease, or a symptom thereof associated with or caused by a fungal infection, the method comprising administering to the subject an isolated or purified antibody or an antigen-binding fragment or an isolated antiserum comprising an antibody, or an antigen-binding fragment thereof, that specifically binds an immunogenic peptide of claim 1 in an amount effective to treat or protect the subject against the fungal disease or a symptom thereof.

8. The method of claim 7, wherein the antibody is a monoclonal antibody, a polyclonal antibody, or an antigen-binding fragment thereof.

9. The method of claim 7, wherein the fungal infection is associated with or caused by an Aspergillus, Candida, Cryptococcus, or Pneumocystis fungal pathogen.

10. The method of claim 7, wherein the disease, or a symptom thereof, is selected from pulmonary disease, asthma, severe asthma, refractory asthma, Chronic Obstructive Pulmonary Disease (COPD), chronic bronchitis, pneumonia, Pneumocystis pneumonia, bronchiectasis, aspergillosis, Invasive Pulmonary Aspergillosis (IPA), vaginitis, urinary tract infections (UTIs), organ transplant, tissue transplant, immunodeficiency disease, HIV, AIDS, HIV/AIDS, congenital disease, autoimmune disease, rheumatoid arthritis, psoriasis, inflammation-related disease, diabetes, Type 1 diabetes, or Type 2 diabetes.

11. The method of claim 7, wherein the antibody or an antigen-binding fragment thereof specifically binds a Kex peptide of one or more of Pneumocystis, Aspergillus, Candida, or Cryptococcus.

12. The method of claim 11, wherein the Aspergillus-associated disease is aspergillosis or Invasive Pulmonary Aspergillosis (IPA) and/or symptoms thereof.

13. The method of claim 7, wherein the subject is immunocompromised or immunosuppressed.

14. The method of claim 13, wherein the immunocompromised or immunosuppressed subject is a pre-transplant subject or a post-transplant subject.

15. The method of claim 14, wherein the immunocompromised or immunosuppressed subject is being treated for cancer, an immunodeficiency disease, a congenital disease, or an autoimmune disease.

16. The method of claim 13, wherein the immunocompromised or immunosuppressed subject is being treated for HIV, AIDS, HIV/AIDS, rheumatoid arthritis, or psoriasis.

17. The method of claim 13, wherein the subject is a human.

18. A vaccine comprising an effective amount of PF-KEX2b peptide of SEQ ID NO: 2 or a polynucleotide encoding PF-KEX2b.

19. A vaccine comprising an effective amount of PF-KEX1b peptide of SEQ ID NO: 1 or a polynucleotide encoding PF-KEX1b.

20. The vaccine of claim 18, wherein the PF-KEX2b peptide of SEQ ID NO: 2 or the PF-KEX1b peptide of SEQ ID NO: 1 is recombinant, recombinantly produced, and/or is isolated.
